SEQUENCE LISTING (89 sequences)

SEQ ID NO: 1 - protein, 15 aa, ARTIFICIAL SEQUENCE
  Peptide analog
ENPVVHFFKNIVTPR

SEQ ID NO: 2 - protein, 15 aa, ARTIFICIAL SEQUENCE
  Peptide analog
FPFNVPTNIVKVERH

SEQ ID NO: 3 - protein, 25 aa, ARTIFICIAL SEQUENCE
  Peptide analog
ENPVVHFFKNIVTPRTPPPSQGKGR

SEQ ID NO: 4 - protein, 15 aa, ARTIFICIAL SEQUENCE
  Peptide analog
EKPKVEAYKAAAAPA

SEQ ID NO: 5 - protein, 10 aa, ARTIFICIAL SEQUENCE
  Peptide analog
KVEAYKAAAA

SEQ ID NO: 6 - protein, 6 aa, ARTIFICIAL SEQUENCE
  Peptide analog
KVEAYK

SEQ ID NO: 7 - protein, 5 aa, ARTIFICIAL SEQUENCE
  Peptide analog
VEAYK

SEQ ID NO: 8 - protein, 15 aa, ARTIFICIAL SEQUENCE
  Peptide analog
EKPKVEAYKAAAAPX

SEQ ID NO: 9 - protein, 15 aa, ARTIFICIAL SEQUENCE
  Peptide analog
EKPKVEAYKAAAAXX

SEQ ID NO: 10 - protein, 15 aa, ARTIFICIAL SEQUENCE
  Peptide analog
EKPKVEAYKAAAXXX

SEQ ID NO: 11 - protein, 15 aa, ARTIFICIAL SEQUENCE
  Peptide analog
EKPKVEAYKAAXXXX

SEQ ID NO: 12 - protein, 15 aa, ARTIFICIAL SEQUENCE
  Peptide analog
EKPKVEAYKAXXXXX

SEQ ID NO: 13 - protein, 15 aa, ARTIFICIAL SEQUENCE
  Peptide analog
EKPKVEAYKXXXXXX

SEQ ID NO: 14 - protein, 15 aa, ARTIFICIAL SEQUENCE
  Peptide analog
EKPKVEAYXXXXXXX

SEQ ID NO: 15 - protein, 15 aa, ARTIFICIAL SEQUENCE
  Peptide analog
EKPKVEAXXXXXXXX

SEQ ID NO: 16 - protein, 15 aa, ARTIFICIAL SEQUENCE
  Peptide analog
EKPKVEXXXXXXXXX

SEQ ID NO: 17 - protein, 15 aa, ARTIFICIAL SEQUENCE
  Peptide analog
EKPKVXXXXXXXXXX

SEQ ID NO: 18 - protein, 15 aa, ARTIFICIAL SEQUENCE
  Peptide analog
EKPKXXXXXXXXXXX

SEQ ID NO: 19 - protein, 15 aa, ARTIFICIAL SEQUENCE
  Peptide analog
EKPXXXXXXXXXXXX

SEQ ID NO: 20 - protein, 15 aa, ARTIFICIAL SEQUENCE
  Peptide analog
EKXXXXXXXXXXXXX

SEQ ID NO: 21 - protein, 15 aa, ARTIFICIAL SEQUENCE
  Peptide analog
EXXXXXXXXXXXXXX

SEQ ID NO: 22 - protein, 15 aa, ARTIFICIAL SEQUENCE
  Peptide analog
XXXXXXXXXXXXXXX

SEQ ID NO: 23 - protein, 15 aa, ARTIFICIAL SEQUENCE
  Peptide analog
XXXXXEAXXXXXXXX

SEQ ID NO: 24 - protein, 10 aa, ARTIFICIAL SEQUENCE
  Peptide analog
KVEAYKAAAX

SEQ ID NO: 25 - protein, 10 aa, ARTIFICIAL SEQUENCE
  Peptide analog
KVEAYKAAXX

SEQ ID NO: 26 - protein, 10 aa, ARTIFICIAL SEQUENCE
  Peptide analog
KVEAYKAXXX

SEQ ID NO: 27 - protein, 10 aa, ARTIFICIAL SEQUENCE
  Peptide analog
KVEAYKXXXX

SEQ ID NO: 28 - protein, 10 aa, ARTIFICIAL SEQUENCE
  Peptide analog
KVEAYXXXXX

SEQ ID NO: 29 - protein, 10 aa, ARTIFICIAL SEQUENCE
  Peptide analog
KVEAXXXXXX

SEQ ID NO: 30 - protein, 10 aa, ARTIFICIAL SEQUENCE
  Peptide analog
KVEXXXXXXX

SEQ ID NO: 31 - protein, 10 aa, ARTIFICIAL SEQUENCE
  Peptide analog
KVXXXXXXXX

SEQ ID NO: 32 - protein, 10 aa, ARTIFICIAL SEQUENCE
  Peptide analog
KXXXXXXXXX

SEQ ID NO: 33 - protein, 10 aa, ARTIFICIAL SEQUENCE
  Peptide analog
XXEAXXXXXX

SEQ ID NO: 34 - protein, 10 aa, ARTIFICIAL SEQUENCE
  Peptide analog
XXXXXXXXXX

SEQ ID NO: 35 - protein, 6 aa, ARTIFICIAL SEQUENCE
  Peptide analog
KVEAYX

SEQ ID NO: 36 - protein, 6 aa, ARTIFICIAL SEQUENCE
  Peptide analog
KVEAXX

SEQ ID NO: 37 - protein, 6 aa, ARTIFICIAL SEQUENCE
  Peptide analog
KVEXXX

SEQ ID NO: 38 - protein, 6 aa, ARTIFICIAL SEQUENCE
  Peptide analog
KVXXXX

SEQ ID NO: 39 - protein, 6 aa, ARTIFICIAL SEQUENCE
  Peptide analog
KXXXXX

SEQ ID NO: 40 - protein, 6 aa, ARTIFICIAL SEQUENCE
  Peptide analog
XXXXXX

SEQ ID NO: 41 - protein, 6 aa, ARTIFICIAL SEQUENCE
  Peptide analog
XXEAXX

SEQ ID NO: 42 - protein, 5 aa, ARTIFICIAL SEQUENCE
  Peptide analog
VEAYX

SEQ ID NO: 43 - protein, 5 aa, ARTIFICIAL SEQUENCE
  Peptide analog
VEAXX

SEQ ID NO: 44 - protein, 5 aa, ARTIFICIAL SEQUENCE
  Peptide analog
VEXXX

SEQ ID NO: 45 - protein, 5 aa, ARTIFICIAL SEQUENCE
  Peptide analog
VXXXX

SEQ ID NO: 46 - protein, 5 aa, ARTIFICIAL SEQUENCE
  Peptide analog
XXXXX

SEQ ID NO: 47 - protein, 5 aa, ARTIFICIAL SEQUENCE
  Peptide analog
XEAXX

SEQ ID NO: 48 - protein, 15 aa, ARTIFICIAL SEQUENCE
  Peptide analog
EKPKVEAYKAAAAPX

SEQ ID NO: 49 - protein, 15 aa, ARTIFICIAL SEQUENCE
  Peptide analog
EKPKVEAYKAAAXPX

SEQ ID NO: 50 - protein, 15 aa, ARTIFICIAL SEQUENCE
  Peptide analog
EKPKVEAYKAAXXPX

SEQ ID NO: 51 - protein, 15 aa, ARTIFICIAL SEQUENCE
  Peptide analog
EKPKVEAYKAXXXPX

SEQ ID NO: 52 - protein, 15 aa, ARTIFICIAL SEQUENCE
  Peptide analog
EKPKVEAYKXXXXPX

SEQ ID NO: 53 - protein, 15 aa, ARTIFICIAL SEQUENCE
  Peptide analog
EKPKVEAYXXXXXPX

SEQ ID NO: 54 - protein, 15 aa, ARTIFICIAL SEQUENCE
  Peptide analog
EKPKVEAXXXXXXPX

SEQ ID NO: 55 - protein, 15 aa, ARTIFICIAL SEQUENCE
  Peptide analog
EKPKVEXXXXXXXPX

SEQ ID NO: 56 - protein, 15 aa, ARTIFICIAL SEQUENCE
  Peptide analog
EKPKVXXXXXXXXPX

SEQ ID NO: 57 - protein, 15 aa, ARTIFICIAL SEQUENCE
  Peptide analog
EKPKXXXXXXXXXPX

SEQ ID NO: 58 - protein, 15 aa, ARTIFICIAL SEQUENCE
  Peptide analog
EKPXXXXXXXXXXPX

SEQ ID NO: 59 - protein, 15 aa, ARTIFICIAL SEQUENCE
  Peptide analog
EXPXXXXXXXXXXPX

SEQ ID NO: 60 - protein, 15 aa, ARTIFICIAL SEQUENCE
  Peptide analog
XXPXXXXXXXXXXPX

SEQ ID NO: 61 - protein, 15 aa, ARTIFICIAL SEQUENCE
  Peptide analog
XXPXXEAXXXXXXPX

SEQ ID NO: 62 - protein, 10 aa, ARTIFICIAL SEQUENCE
  Peptide analog
KVEAYKAAAX

SEQ ID NO: 63 - protein, 10 aa, ARTIFICIAL SEQUENCE
  Peptide analog
KVEAYKAAXX

SEQ ID NO: 64 - protein, 10 aa, ARTIFICIAL SEQUENCE
  Peptide analog
KVEAYKAXXX

SEQ ID NO: 65 - protein, 10 aa, ARTIFICIAL SEQUENCE
  Peptide analog
KVEAYKXXXX

SEQ ID NO: 66 - protein, 10 aa, ARTIFICIAL SEQUENCE
  Peptide analog
KVEAYXXXXX

SEQ ID NO: 67 - protein, 10 aa, ARTIFICIAL SEQUENCE
  Peptide analog
KVEAXXXXXX

SEQ ID NO: 68 - protein, 10 aa, ARTIFICIAL SEQUENCE
  Peptide analog
KVEXXXXXXX

SEQ ID NO: 69 - protein, 10 aa, ARTIFICIAL SEQUENCE
  Peptide analog
KVXXXXXXXX

SEQ ID NO: 70 - protein, 10 aa, ARTIFICIAL SEQUENCE
  Peptide analog
KXXXXXXXXX

SEQ ID NO: 71 - protein, 10 aa, ARTIFICIAL SEQUENCE
  Peptide analog
XXXXXXXXXX

SEQ ID NO: 72 - protein, 10 aa, ARTIFICIAL SEQUENCE
  Peptide analog
XXEAXXXXXX

SEQ ID NO: 73 - protein, 6 aa, ARTIFICIAL SEQUENCE
  Peptide analog
KVEAYX

SEQ ID NO: 74 - protein, 6 aa, ARTIFICIAL SEQUENCE
  Peptide analog
KVEAXX

SEQ ID NO: 75 - protein, 6 aa, ARTIFICIAL SEQUENCE
  Peptide analog
KVEXXX

SEQ ID NO: 76 - protein, 6 aa, ARTIFICIAL SEQUENCE
  Peptide analog
KVXXXX

SEQ ID NO: 77 - protein, 6 aa, ARTIFICIAL SEQUENCE
  Peptide analog
KXXXXX

SEQ ID NO: 78 - protein, 6 aa, ARTIFICIAL SEQUENCE
  Peptide analog
XXXXXX

SEQ ID NO: 79 - protein, 6 aa, ARTIFICIAL SEQUENCE
  Peptide analog
XXEAXX

SEQ ID NO: 80 - protein, 5 aa, ARTIFICIAL SEQUENCE
  Peptide analog
VEAYX

SEQ ID NO: 81 - protein, 5 aa, ARTIFICIAL SEQUENCE
  Peptide analog
VEAXX

SEQ ID NO: 82 - protein, 5 aa, ARTIFICIAL SEQUENCE
  Peptide analog
VEXXX

SEQ ID NO: 83 - protein, 5 aa, ARTIFICIAL SEQUENCE
  Peptide analog
VXXXX

SEQ ID NO: 84 - protein, 5 aa, ARTIFICIAL SEQUENCE
  Peptide analog
XXXXX

SEQ ID NO: 85 - protein, 5 aa, ARTIFICIAL SEQUENCE
  Peptide analog
XEAXX

SEQ ID NO: 86 - protein, 15 aa, ARTIFICIAL SEQUENCE
  Peptide analog
XXPXXXXXXXXXXPX

SEQ ID NO: 87 - protein, 15 aa, ARTIFICIAL SEQUENCE
  Peptide analog
XXPXXXXXXXXXXPX

SEQ ID NO: 88 - protein, 10 aa, ARTIFICIAL SEQUENCE
  Peptide analog
XXXXXXXXXX

SEQ ID NO: 89 - protein, 10 aa, ARTIFICIAL SEQUENCE
  Peptide analog
XXXXXXXXXX

(19) United States
(12) Patent Application Publication
(10) Pub. No.: US 2014/0348861 A1
(43) Pub. Date: Nov. 27, 2014

(54) SYNTHETIC PEPTIDES AND RANDOM COPOLYMERS FOR THE TREATMENT OF AUTOIMMUNE DISORDERS

(71) Applicants: Avadhesha Surolia, New Delhi (IN); Ravi Kant Gautam, New Delhi (IN); Vishnu Kumar Dwivedi, New Delhi (IN); Sarika Gupta, New Delhi (IN)

(72) Inventors: Avadhesha Surolia, New Delhi (IN); Ravi Kant Gautam, New Delhi (IN); Vishnu Kumar Dwivedi, New Delhi (IN); Sarika Gupta, New Delhi (IN)

(73) Assignee: NATIONAL INSTITUTE OF IMMUNOLOGY, New Delhi (IN)

(21) Appl. No.: 14/115,598

(22) PCT Filed: May 3, 2012
(86) PCT No.: PCT/IB2012/000864

§ 371 Date: Nov. 4, 2013

(30) Foreign Application Priority Data
May 5, 2011 (IN) 1313/DEL/2011

(51) Int. Cl. C07K 14/00; C07K 7/08; C07K 7/06

(52) U.S. Cl. CPC C07K 14/001; C07K 7/06; C07K 7/08
USPC 424/184.1; 530/350; 435/188; 530/330; 530/329; 530/328; 530/326; 530/324

(57) ABSTRACT

Synthetic peptides and peptide copolymers for amelioration of autoimmune neurological syndrome, inflammatory and/or demyelinating conditions such as encephalomyletis are provided herein. The synthetic peptides and peptide copolymers as disclosed are obtained by substitution of at least one alpha amino acid by beta amino acid and/or β3-homo amino acid.

### FIELD OF INVENTION

The present invention relates to synthetic peptides and random copolymers (random peptides) for treatment of autoimmune and/or demyelinating conditions such as multiple sclerosis (MS).

### BACKGROUND OF THE INVENTION

Multiple sclerosis (MS) is a complex autoimmune neurological syndrome characterized by the presence of inflammatory cells and demyelinating lesions in the white matter of brain and spinal cord. It is a debilitating disease, which usually starts in young adulthood. In majority of the cases (˜85%) the disease initially manifests in a relapsing remitting form, RRMS, which eventually progresses into an irreversible form, known as secondary progressive MS or SPMS (Hemmer B, Archelos J J, Hartung H P. New concepts in the immunopathogenesis of multiple sclerosis. Nat Rev Neurosci. 2002 April; 3(4):291-301). There are about 1.3 million people affected worldwide with the disease (WHO, Multiple Sclerosis International Federation. Atlas multiple sclerosis resources in the world, 2008). It occurs with two times greater frequency in women than in men. Body's own immune system is considered to be the key player in the initiation and the progression of the disease process. Studies so far establish the role of an autoreactive T cell repertoire in mediating self-destruction. The helper T cells (CD4+) provide the required microenvironment to the cytotoxic T cells (CD8+) in the central nervous system (CNS) that eventually destroy the insulating myelin sheath of the white matter neurons in CNS (Steinman L. Multiple sclerosis: a coordinated immunological attack against myelin in the central nervous system. Cell. 1996 May 3; 85(3):299-302; Huseby E S, Liggitt D, Brabb T, Schnabel B, Ohlén C, Goverman J. A pathogenic role for myelin-specific CD8 (+) T cells in a model for multiple sclerosis. J Exp Med. 2001 Sep. 3; 194 (5):669-76). A genetic basis for the occurrence of the disease is apparent from the prevalence of MS in individuals with certain haplotypes of MHC I and II such as HLADR15, HLADR51 (encoded by HLADRB1*1501, HLADRB5*0101 alleles) and HLA-A3, HLA-B7 (encoded by HLA-A*0103, HLA-B*0707) (Fogdell-Hahn A, Ligers A, Grønning M, Hillert J, Olerup O. Multiple sclerosis: a modifying influence of HLA class I genes in an HLA class II associated autoimmune disease. Tissue Antigens. 2000 February; 55 (2): 140-8; Harbo H F, Lie B A, Sawcer S, Celius E G, Dai K Z et al. Genes in the HLA class I region may contribute to the HLA class II-associated genetic susceptibility to multiple sclerosis. Tissue Antigens. 2004 March; 63(3):237-47; Friese M A, Fugger L. Autoreactive CD8+ T cells in multiple sclerosis: a new target for therapy? Brain, 2005 August; 128(Pt 8):1747-63. Epub 2005 Jun. 23. Review. Erratum in: Brain. 2005; 128: 2215).

Since, autoimmune diseases arise from aberrant immune reactions, consequently, traditional therapeutics, so far, have focused either on immune suppression or on impairment of immune surveillance. Such therapeutic agents initially seemed assuring as a potential therapy but over a period of time have turned out to be associated with severe complications occurring as a result of generalized immune suppression. Thus, effective antigen specific therapies that target only the autoimmune component are currently gaining currency. Auto reactive CD4+ cells are established players in etiopathogenesis of MS, hence antigen specific approaches should be and have been aimed at suppressing their activation. The role of CD8+ T cells in disease process has been highlighted recently. Thus, both CD4+ and CD8+ T cells need to be considered while designing appropriate treatment strategies in near future. In many cases, such therapies act at the level of antigen presentation. They interfere with the physiological process involved in display of myelin derived auto antigens to the auto reactive T cells hence blocking their activation or inducing antigen specific tolerance (Lutterotti A, Sospedra M, Martin R. Antigen-specific therapies in MS—Current concepts and novel approaches. J Neurol Sci. 2008; 274(1-2):18-22).

Copolymer 1, popularly known as Glatiramer Acetate (GA) or Copaxone or Glatimer is an established representative of such class of drugs and is the only FDA approved therapeutic peptide being currently used for the treatment of MS in humans without many side effects. Glatiramer Acetate is a synthetic random copolymer (polypeptide), an analog of myelin basic protein (MBP), which is a natural component of the myelin sheath. It is a random copolymer composed of four naturally occurring amino acids namely L-tyrosine (Y), L-glutamic acid (E), L-alanine (A) and L-lysine (K) in a molar ratio of 5, 3, 1.5 and 1 respectively. The average molecular weight is 4,700-11,000 Daltons. Upon degradation in-vivo, it essentially releases smaller active peptide fragments which compete with myelin antigens implicated in autoimmune demyelinating diseases (e.g. multiple sclerosis) such as MBP (Myelin Basic Protein), PLP (Proteolipid Protein) and MOG (Myelin Oligodendrocyte Glycoprotein) for binding to HLA DR2 (class II MHC) molecules on the surface of antigen presenting cells and is therefore, used for the suppression of demyelinating disease in both experimental animals (EAE) and humans (relapsing remitting form of MS).

U.S. Pat. No. 3,849,550 describes a composition for use in the treatment or prevention of experimental allergic encephalomyelitis comprising a synthetic water soluble co-polymer comprising in combination alanine, glutamic acid, lysine and tyrosine.

U.S. Pat. Nos. 6,048,898; 5,800,808; 5,981,589 and 3,849,550 describes the process for the preparation of copolymer 1 (Glatiramer Acetate). They all employ as starting materials four N-carboxyanhydrides derived from alanine, γ-benzyl glutamate, N.sup.epsilon.-trifluoroacetyl lysine and tyrosine.

GA, acts principally by polarizing the immune response towards an anti-inflammatory phenotype i.e. Th2 and by inducing a regulatory T cell population (Vieira P L, Heystek H C, Wormmeester J, Wierenga E A, Kapsenberg M L. It also (copolymer-1, copaxone) promotes Th2 cell development and increased IL-10 production through modulation of dendritic cells. J. Immunol. 2003; 170(9):4483-8; Amon R, Aharoni R. Mechanism of action of glatiramer acetate in multiple sclerosis and its potential for the development of new applications. Proc Natl Acad Sci USA. 2004; 101:14593-8). Though widely used, the success rate of GA in reducing the relapses is only 30%.

Besides GA, several other copolymers, keeping in view the key contact residues between HLA DR2 (a HLA haplotype most commonly associated with MS) and MBP (85-99; immunodominant epitope of MBP; a natural ligand of HLA DR2), have been formulated and tested in experimental animals. The most noteworthy among the synthesized copolymers have been F (L-Phenylalanine), Y (L-Tyrosine), A (L-Alanine), K (L-Lysine) and V (L-Valine), W (L-Tryptophan), A (L-Alanine), K (L-Lysine) (Fridkis-Hareli M et al., Novel synthetic amino acid copolymers that inhibit autoantigen specific T-cell responses and suppress experimental autoimmune encephalomyelitis. J Clin Invest. 2002; 109(12): 1635-1643; Stern J N et al. Amelioration of proteolipid protein 139-151-induced encephalomyelitis in SJL mice by modified amino acid copolymers and their mechanisms. Proc Natl Acad Sci USA. 2004; 101(32):11743-8; Illés Z et al Modified amino acid copolymers suppress myelin basic protein 85-99-induced encephalomyelitis in humanized mice through different effects on T cells. Proc Natl Acad Sci USA. 2004; 101(32):11749-54). These copolymers were designed to have an optimized binding with HLA DR2 which was lacking in cop1 (GA, YEAK). The amino acids forming copolymer 1 possess certain features that make them slightly less suitable when the binding pocket of HLA DR2 is considered for e.g. tyrosine (Y) in YEAK has a bulky —R group which would not fit properly into the small P1 pocket of HLA DR2, alanine (A) is too small while glutamic acid (E) and lysine (K) are too hydrophilic. So, the new copolymers were tailored to include phenylalanine (F) in place of glutamic acid (E) in FYAK and both tyrosine (Y) and glutamic acid (E) were replaced with valine (V) and tryptophan (W) in VWAK to fit better into the pocket P1 of HLA DR2. Modified random copolymers such as FYAK have been reported to have superior therapeutic efficacy than GA (Fridkis-Hareli M et al Novel synthetic amino acid copolymers that inhibit autoantigen specific T-cell responses and suppress experimental autoimmune encephalomyelitis. J Clin Invest. 2002; 109(12): 1635-1643). After successful preclinical testing, phase Ib clinical trials are in progress for FYAK copolymer as reported by Peptimmune.

Another related class of therapeutics for autoimmune disorders constitutes altered peptide ligands (APL) known to exert their suppressive effect on clinical progression of an autoimmune condition by inducing anergy in autoreactive T-cells by suboptimal signaling through T cell receptor (TCR). In an altered peptide ligand of MBP (87-99), APL A91 (NBI-5788), lysine (K), a major T cell contact residue has been replaced with alanine (A). Substitution of K with A results into impaired signaling through TCR on autoreactive T cells thus making them anergic (Gaur A. Amelioration of relapsing experimental autoimmune encephalomyelitis with altered myelin basic protein peptides involves different cellular mechanisms, Journal of Neuroimmunology. 1997; 74(1-2): 149-158) After initial encouraging results with APL A91 (NBI-5788) further clinical studies have been abandoned after it failed to meet its primary end point (Neurocrine Biosciences).

Another peptide molecule, an analog of MBP (85-99), J5 (Stern J N, Illés Z, Reddy J, Keskin D B, Fridkis-Hareli M et al. Peptide 15-mers of defined sequence that substitute for random amino acid copolymers in amelioration of experimental autoimmune encephalomyelitis. Proc Natl Acad Sci USA. 2005; 102(5):1620-5; Strominger J L and Fridkis-Hareli M. Therapeutic peptides for demyelinating conditions. U.S. Pat. No. 7,456,252 B2), has shown promise as an effective antagonist for binding of MBP (85-99) to HLA DR2, but was found to have moderate efficacy in mice with experimental autoimmune encephalomyelitis (EAE).

Of the therapeutics or potential therapeutics belonging to the class of therapeutic molecules which bring antigen specific immune suppression have been observed to have limited therapeutic activity in biological systems which can be attributed to their limited half life and/or inefficient uptake or presentation in-vivo.

U.S. Pat. No. 5,948,764 describes peptide analogs at least 7 residues long derived from MBP (87-99). The residues at position 87, 88, 97, 98, 99 are changed to D-amino acids. The peptides inhibit binding of MBP (86-99) to rat spleen cells. Peptide analogs mentioned above suppresses MBP (87-99) induced EAE.

U.S. Pat. No. 6,740,638 describes peptide analogues of human myelin basic protein containing residues 87-99 are provided. Residue 91 of the peptide analogues is altered from the L-lysine residue found in the native protein to any other amino acid. The peptides as described are analogues of human MBP (87-99) where residue 91 is altered from L-lysine to L-alanine.

U.S. Pat. Nos. 6,930,168 and 7,456,252 describes peptide analogs, including J5 (SEQ ID NO. 93) of MBP (85-99) and peptides containing two tyrosines and one lysine or one tyrosine, valine and lysine. Peptide analogs mentioned above bind to HLA DR2 and block the binding of MBP (85-99) or GA (cop1) to HLA DR2. Additionally peptide analogs also suppress the activation of MBP (85-99) specific HLA DR2 restricted T cell hybridoma such as Hy1B or 8073. The peptides as described are proposed to be useful for the treatment of demyelinating conditions.

U.S. Pat. Appl. No. US2007/0264229 describes non random peptide analogs MBP (85-99), including J5 (SEQ ID NO. 5) and others which contain two tyrosines (Y) and one lysine (K) or one tyrosine (Y), valine (V) and lysine (K). The peptides as described inhibit binding of MBP (85-99) to HLA DR2 more strongly than GA (cop1) thus blocking the presentation of MBP (85-99). The above mentioned peptides also inhibits IL-2 production (activation) of MBP (85-99) specific HLA DR2 restricted T cell hybridoma. Peptides mentioned above suppress MBP (85-99) induced EAE in humanized mice (mice expressing human HLADR2 and MBP (85-99) specific HLA DR2 restricted T cell receptor). Peptides mentioned above suppress PLP (131-151) induced EAE in SJL/J mice. Peptides (mentioned above) specific T cells have ability to suppress EAE induced using PLP (131-151) in SJL/J mice. The peptides are immunogenic thus treatment with them results in increased frequencies of Th2 cells specific to that particular peptide, which produce anti-inflammatory cytokines (IL-4 and IL-10). However, the peptides do not stimulate MBP (85-99) or PLP specific T cells.

U.S. Pat. Appl. No. 2009/0214580 describes complex peptide mixtures with defined sequences in comparison to GA which is a random copolymer of tyrosine (Y), glutamic acid (E), alanine (A) and lysine (K). In other words complex peptide mixture as described is a multimer of a peptide with sequence AEKY. The application further describes that composition and peptide length affects the ability of complex peptide mixtures to stimulate PBMCs from MS patients when compared to GA. Like GA, complex peptides mixtures are also cross reactive to myelin antigens thus are able to bring bystanders suppression once they encounter myelin antigens. Pretreatment with complex peptide mixtures can suppress PLP (131-151) induced EAE.

Most of the peptide therapeutics described in the prior art have serious problems associated with them (common to all peptide therapeutics) which affect their efficacy significantly such as their very limited biological half life and poor uptake/presentation by antigen presenting cells. Additionally some of the peptide therapeutics which showed promise (altered peptide ligands) exhibited serious side effects upon administration to a subject in need thereof. In view of the problems associated with the existing treatment options available for autoimmune, demyelinating conditions such as MS, there is an undeniable need for providing an effective therapy and therapeutic agent for the treatment of autoimmune demyelinating conditions.

### SUMMARY OF THE INVENTION

An aspect of the present invention is to provide a synthetic peptide for amelioration of a demyelinating disorder comprising at least 5 amino acids with valine at position P1, tyrosine at position P4 and lysine at position P5, wherein the peptide consists of at least one β-amino acid and/or β³-homo amino acid.

Another aspect of the present invention is to provide a synthetic random copolymer (random peptides) of
tyrosine, glutamic acid, alanine and lysine, or
tyrosine, phenylalanine, alanine and lysine, or
tryptophan, valine, alanine and lysine
   wherein alanine is β-alanine (A_{β}) and/or β-homoalanine (A_{β}³); lysine is β-lysine (K) and/or β-homolysine (K_{β}³), tyrosine is β-tyrosine (Y_{β}) and/or β-homotyrosine (Y_{β}³); valine is β-valine (V_{β}) and/or β-homovaline (V_{β}³); glutamic acid is β-glutamic acid (E_{β}) and/or β-homoglutamic acid (E_{β}³); phenylalanine is β-phenylalanine and/or β-homophenylalanine (F_{β}³) and tryptophan is β-tryptophan (W_{β}) and/or β-homotryptophan (W_{β}³).

Another aspect of the present invention is to provide a composition for amelioration of a demyelinating disorder, wherein said composition comprises a) a plurality of the synthetic peptides comprising at least 5 amino acids and having valine at position P1, tyrosine at position P4 and lysine at position P5, wherein the peptide consists of at least one β-amino acid and/or β³-homo amino acid, or b) a plurality the synthetic random copolymer of tyrosine, glutamic acid, alanine and lysine, or tyrosine, phenylalanine, alanine and lysine, or tryptophan, valine, alanine and lysine or c) a combination of (a) and (b); wherein alanine is (β-alanine (A_{β}) and/or β-homoalanine (A_{β}³), lysine is β-lysine (K_{β}) and/or β-homolysine (K_{β}³), tyrosine is β-tyrosine (Y_{β}) and/or β-homotyrosine (Y_{β}³); valine is β-valine (V_{β}) and/or β-homovaline (V_{β}³); glutamic acid is β-glutamic acid (E_{β}) and/or β-homoglutamic acid (E_{β}³); phenylalanine is β-phenylalanine (F_{β}) and/or β-homophenylalanine (F_{β}³) and tryptophan is β-tryptophan (W_{β}) and/or β-homotryptophan (W_{β}³).

### BRIEF DESCRIPTION OF ACCOMPANYING DRAWINGS

FIG. 1 shows a set of line graphs depicting proliferative responses (incorporation ³[H]-thymidine) of CD4+ (a) and CD8+ (b) T-cells from animals immunized with MBP (85-109, SEQ ID NO: 3) and pre-treated with GA, J5 (SEQ ID NO: 2), S27 (SEQ ID NO: 32) when co-cultured with spleen derived dendritic cells (SPDCs) pulsed with increasing concentrations of MBP (85-109, SEQ ID NO:3) or purified protein derivative (PPD).

FIG. 2 depicts

(a) the therapeutic efficacies of GA, J5 (SEQ ID NO: 4) and various MBP analogs as set forth in SEQ ID NO: 5 to 89 (J5a, J5b, J5c, S1, S2, S3, S4, S5, S6, S7, S8, S9, S10, S11, S12, S13, S14, S15, S16, S17, S18, S19, S20, S21, S22, S23, S24, S25, S26, S27, S28, S29, S30, S31, S32, S33, S34, S35, S36, S37, S38, S39, S40, S41, S42, S43, S44, S45, S46, S47, S48, S49, S50, S51, S52, S53, S54, S55, S56, S57, S58, S59, S60, S61, S62, S63, S64, S65, S66, S67, S68, S69, S70, S71, S72, S73, S74, S75, S76, S77, S78, S79, S80, S81 and S82);
(b) therapeutic effect of S15 (SEQ ID NO: 22), S27 (SEQ ID NO: 34), S15+S27 (combination) in comparison to GA and J5 (SEQ ID NO: 4);
(c) (d) dosage kinetics of S27 (SEQ ID NO: 34);
(e) (f) therapeutic efficacies of S27 (SEQ ID NO: 34) in comparison to GA and J5 (SEQ ID NO: 4) in C57BL6/J mice with chronic EAE;
(g) (h) (i) prophylactic efficacies of GA, J5 (SEQ ID NO: 4) and S27 (SEQ ID NO: 34) in SJL/J mice with relapsing remitting EAE;
(j) (k) prophylactic efficacies of GA, J5 (SEQ ID NO: 4) and S27 (SEQ ID NO: 34) in C57BL6/J mice with chronic EAE. Therapeutic or prophylactic efficacies have been demonstrated in terms of reduction in disability score/cumulative disability score and delay in clinical onset of disease (prophylactic group).

FIG. 3 comprises a set of horizontal bar diagrams demonstrating percent inhibition of binding of biotinylated MBP (85-99) to HLA DR2 by 5 μM MBP (85-99, Seq ID no. 1), scrambled MBP (85-99, Seq ID no. 2), GA, S27 (Seq ID no. 34).

FIG. 4 shows a set of bar diagram depicting the levels of IFNg, IL-2, IL-4 and IL-10 in the culture supernatants of spleenocytes isolated from the various experimental groups viz disease control, GA, J5 (SEQ ID NO: 4) and S27 (SEQ ID NO: 34) treated groups at the four weeks stimulated with respective peptides for 48 h.

FIG. 5 comprises a set of horizontal bar diagrams demonstrating percent inhibition of binding of biotinylated MBP (85-99) to HLA DR2 by 5 μM MBP (85-99, SEQ ID NO: 1), scrambled MBP (85-99, SEQ ID NO: 2), GA, J91, J92, S101, S102, S103.

FIG. 6 depicts the therapeutic activity of various random copolymers namely GA, J91, J92, S101, S102, S103. Therapeutic or prophylactic activity has been demonstrated in terms of reduction in cumulative disability score.

FIG. 7 shows a set of bar diagram depicting the levels of IFN-g, IL-2, IL-4 and IL-10 in the culture supernatants of spleenocytes isolated from the various experimental groups viz. disease control, GA, J91, J92, S101, S102, S103 treated groups at the four weeks stimulated with respective random copolymers for 48 h.

### DETAILED DESCRIPTION OF THE INVENTION

The present invention provides synthetic peptides and random copolymers (random peptides) for amelioration of autoimmune, inflammatory and/or demyelinating neurological syndrome such as encephalomyletis, wherein said peptides are derived from myelin basic protein (MBP 85-99) and J5; and said random copolymers are derived from GA. The synthetic peptides as disclosed are obtained by modification of at least one amino acid residue in MBP 85-99 peptide or its analog such as J5, wherein said modification comprises substitution of at least one alpha amino acid by β-amino acid and/or β³-homo amino acid, and the synthetic peptides comprise at least 5 amino acids containing valine at position P1, tyrosine at position P4 and lysine at position P5. The synthetic peptides thus obtained exhibits increased binding affinity to class I and/or class II MHCs relative to the MBP 85-99 peptide or it's analog and is capable of blocking the binding of myelin basic protein (MBP) peptide to class I and/or class II MHC.

The present invention herein provides synthetic peptide which is analogs of MBP (85-99) and synthetic random copolymers having significantly improved efficacy for the treatment of autoimmune, demyelinating condition such as MS.

The present invention discloses use of β-amino acids and/or β³-homo amino acids (Table 1) in exogenous therapeutic peptides as a novel strategy to enhance their presentation cross presentation in particular by antigen presenting cells in-vivo or ex-vivo. Additionally, peptide analogs of immuno-dominant epitope of myelin basic protein (MBP), MBP (85-99) containing β-amino acids and/or β³-homo amino acids are being provided treatment with which effectively suppresses or ameliorates the progression of relapsing remitting (RR) or chronic progressive (CP) experimental autoimmune encephalomyelitis (EAE) in SJL/J or C57BL6/J mice by down modulating the presentation of myelin antigens.

Further where exogenous therapeutic peptide is an altered peptide ligand derived from multiple sclerosis associated immunodominant epitope from human myelin basic protein (MBP 85-99), rheumatoid arthritis associated human type II collagen (CII 259-275), human glucose phosphate isomerase (hGPI 325-339), type I diabetes associated human insulin B chain (B9-23), myasthenia gravis associated human acetyl choline receptor alpha-subunit (p195-212, p259-271).

Despite being an excellent inhibitor of MBP (85-99), a moderate therapeutic efficacy of J5 can be attributed to an inherent problem associated with peptide based immuno-therapeutics i.e their low biological half life, inefficient uptake and subsequent presentation by antigen presenting cells (APCs). A solution to the existing problem of lack of therapeutic molecule, compound or agent for treatment of autoimmune, inflammatory and/or demyelinating neurological syndrome such as encephalomyletis was addressed in the present invention by providing the peptide analogs obtained by modifying the amino acid content of MBP (85-99) or J5 peptide by substituting at least one α-amino acid residue with β-amino acid or β³-homoamino acids. In β³-homoamino acid or β-amino acid residues the amino group is attached to the β carbon atom instead to the α carbon atom. Most notable property of β-peptides known is their ability to form amphipathic helix for which longer peptide backbone can be accounted. Formation of amphipathic helix in β-peptides is known to increase their thermodynamic stability and to impart them resistance to proteolytic cleavage which is widely acknowledged (Frackenpohl J, Arvidsson P I, Schreiber J V, Seebach D. The outstanding biological stability of β- and γ-peptides toward proteolytic enzymes: an in vitro investigation with fifteen peptidases. Chembiochem. 2001 Jun. 1; 2(6):445-55; Gademann K, Hintermann T, Schreiber J V. Beta-peptides: twisting and turning. Curr Med Chem. 1999; 6(10):905-25).

It has been surprisingly found that the synthetic peptides and random copolymers (peptides) as disclosed in the present invention having β-amino acid(s) and/or β³-homo amino acid resulted into its enhanced presentation with class I and/or II MHC molecules and thereby effective down modulation of presentation of myelin antigens to myelin reactive CD4+ and/or CD8+ T-cells. This eventually resulted into decreased priming of myelin reactive T-cells, decreased infiltration into CNS. Thus, the synthetic peptides and random copolymers disclosed in the present invention are much more efficacious, stable having longer thermodynamic or biological half life and are capable to sail through cell membranes passively, enter into various “cellular compartments” for example endoplasmic reticulum, late endosomes, trans golgi network and/or class II MHC loading compartment (MIIC), which is required to be available for long duration in the diseased subjects.

The synthetic peptides and the random copolymers as disclosed in the present invention are able to get localized in various cellular compartments by traversing through plasma membranes passively in a receptor independent manner and is efficiently presented and/or cross presented with class I MHC molecules on the surface of antigen presenting cells (APCs) such as macrophages, dendritic cells (DC), spleen derived dendritic cells (SPDC), langerhans cells, microglial cells, etc.

Further, the synthetic peptides and the random copolymers as disclosed in the present invention down modulate the presentation of myelin antigens in association with class I and/or class II MHC molecules, wherein a myelin antigen could be any of the following: myelin basic protein (MBP), proteolipid protein (PLP) or myelin oligodendrocyte glycoprotein (MOG) and autoantigen is a C-terminal region of MBP e.g. MBP (85-99).

The synthetic peptides and the random copolymers as disclosed in the present invention compete efficiently with myelin antigen derived epitopes for binding to antigen binding groove of multiple sclerosis (MS) associated class I and/or class II MHC haplotypes e.g. HLA DR2 (class II MHC) and HLA3 (class I MHC). In other words the peptides down modulate the presentation of myelin antigens by APCs. Also the peptides disclosed are retained for longer duration on the surface of antigen presenting cells bearing MS associated MHC haplotypes.

Further it was found that treatment with the synthetic peptides and/or synthetic random copolymers as disclosed in the present invention results in decreased frequency of myelin reactive cells in central nervous system (CNS) or peripheral lymphoid tissues. In certain embodiment where myelin reactive cells mentioned above for example has Th1, Th17 and/or Th23, CD4+, CD8+, B-cell, NK cell phenotype. Treatment results into increased occurrence of peptide reactive Th2, regulatory T cells, regulatory B cells in CNS or peripheral lymphoid tissue.

The present invention also provides a therapeutic formulation comprising at least one of the synthetic peptides as disclosed in the present invention or their homo-polymers or co-polymers for the treatment of an autoimmune, inflammatory, demyelinating condition in experimental animals or in human subjects at a therapeutically effective dosage, wherein the autoimmune demyelinating condition in human subjects is multiple sclerosis (MS), wherein affected human subject displays any of the four subtypes of MS i.e. relapsing remitting MS (RRMS), secondary progressive MS (SPMS), primary progressive MS (PPMS) or chronic progressive MS (CPMS), symptoms include impaired neuromuscular co-ordination, optic neuritis, bowel dysfunction, or dysregulation of body temperature.

The therapeutic formulation comprising at least one of the synthetic peptides or random copolymers (peptides) as disclosed in the present invention is administered through any of the routes such as subcutaneous, oral, epicutaneous, intradermal, intramuscular, intravenous, intraperitoneal, intrathecal, intracranial in a pharmaceutically acceptable carrier.

In yet another embodiment provides a therapeutic formulation in the form of a kit comprising at least one synthetic peptide and/or random copolymer as its indispensable component. The peptides mentioned above are presented in a form which could be soluble and monomeric, insoluble aggregates, oligomeric or multimeric, wherein the oligomerisation or multimerisation is facilitated by changes in temperature, pH, buffer composition and/or incorporation of amyloidogenic motifs.

The synthetic peptide and/or the random copolymer can be administered once the definitive neurological symptoms appear (treatment), before immunization (prevention) or simultaneously (co-immunization) into experimental animal or human subjects with MS.

The synthetic peptides and copolymers viz. S1-S82, S101-S109 can be used in combination with a known therapy for example environmental enrichment, physiotherapy and acupuncture; and/or known therapeutic for example glatiramer acetate (GA), IFN β, anti VLA-4 (Tysabri), FTY720 (Geneliya) and NBQX.

In a related embodiment of the present invention the therapeutic agent is a random copolymer comprising key residues e.g. L-valine, L-lysine, L-tyrosine, L-glutamic acid, L-tyrosine and L-alanine involved in interactions of myelin antigen derived epitopes with relevant MHCs and T-cell receptor. Further at least one of the amino acids as mentioned is a β³-homo amino acid or their close relatives i.e. β-amino acids.

In a related embodiment as provided in the present invention, a therapeutic formulation consisting any of the peptides or random copolymers wherein any of the amino acid in the peptides is substituted by its analog, where substituted analog is a D-amino acid, is a derivative of parent amino acid where derivatization can be a substitution/addition/modification with chemical entities/functional groups having similar charge and/or size properties such as alkyl, alkenyl, aryl, formyl, phosphate, acetyl, t-butoxyl, halogens e.g. R group of valine (—CH(CH₃)₂) is replaced with —X(CH₃)₂ where X denote any heteroatom (N,O,S).

In another embodiment any of the peptide or copolymer disclosed in the present invention i.e. S1-S82, S101-S109 is modified at —C, —N or both termini with the addition of chemical entities such as —RCO where R is Φ, alkyl. Additionally where there is a substitution and/or addition of few small sized neutral amino acids and/or their analogs to C—, N— or both termini and/or penultimate positions at either or both ends, where neutral small sized amino acid could be glycine, alanine or proline.

In yet another embodiment the β-peptide/peptide backbone is replaced with a homologous or analogous structural entity which forms an amphipathic helix and which may include replacement of one or more peptide bond with a non-peptide bond that is selected from a group consisting of —CS—NH—, —NH—CO— (inverse peptide bond), —CH₂—NH—, —CH₂—S—, —CH₂—CH₂—, —CH═CH—, —CO—CH₂, —CH(OH)CH₂— and —CH₂SO—.

In another embodiment of the present invention the synthetic peptides or copolymers (peptides) exerts their therapeutic effect through enhanced presentation and/or cross presentation of a therapeutic peptide or copolymers and thereby down modulate the presentation of myelin antigens.

The synthetic peptides S1-S82, S101-S109 with modification at C—, N—, or both termini with addition of cell penetrating peptides or motifs either attached covalently, non-covalently and/or separated by linker(s) consisting of a sequence recognized and cleaved by a cell resident protease or peptidase, wherein the cell penetrating peptide could be HIV-1 Tat, penetratin (Antp), poly-lys, poly-arg, MPG, Pep-1, CADY, TP, TP10, transportan, VP22, model amphipathic peptide (MAP) and linker is RVKR sensitive to trans-golgi network, resident endopeptidase furin.

The synthetic peptides as disclosed in the present invention or random copolymers S1-S82, S101-S109 of the present invention exercise their effects due to enhanced bioavailability, ability to cross blood brain barrier and exert its effect in-situ.

The synthetic peptides as disclosed in the present invention or random copolymers S1-S82, S101-S109 as disclosed in the present invention are much more effective than J5, J5a, J5b, J5c or GA when administered through oral route.

The synthetic peptides or random copolymers as disclosed in the present invention exerts its therapeutic effects by polarizing Th1-Th2 response towards Th2, wherein Th1 cells are marked by their ability to produce a group of cytokines such as IFN-gamma, IL-2, IL-6, IL-12, TNF-alpha and Th2 cells are marked by their ability to produce a group of cytokines such as IL-4, IL-10 and IL-13.

The synthetic peptides as disclosed in the present invention exerts its therapeutic effects exerts its therapeutic effects by reducing glutamate cytotoxicity in the central nervous system.

An embodiment of the present invention provides the use of β-amino acids in exogenous peptides as a strategy to enhance their presentation and/or cross presentation in particular. Further in a certain embodiment where β-amino acids are replaced by their close relatives such as β³-homo amino acids (β-substituted-β-homo amino acids) or their isomer or stereoisomer such as those having D-, L-, R-, S-configurations.

Another embodiment of the present invention provides the peptide selected from the group consisting of SEQ ID NO: 8 to SEQ ID NO: 89, preferably SEQ ID NO: 10 to SEQ ID NO: 23; SEQ ID NO: 26 to SEQ ID NO: 34; SEQ ID NO: 39 to SEQ ID NO: 41; SEQ ID NO: 46 to SEQ ID NO: 47; SEQ ID NO: 50 to SEQ ID NO: 56; SEQ ID NO: 64 to SEQ ID NO: 68; SEQ ID NO: 86 to SEQ ID NO: 89, wherein the peptide is able to get localized in various cellular compartments by traversing through plasma membranes passively in a receptor independent manner and is efficiently presented and/or cross presented with class I MHC molecules on the surface of antigen presenting cells (APCs).

In a certain embodiment of the present invention where the peptide mentioned above, down modulates the presentation of myelin antigens in association with class I and/or class II MHC molecules. In a further embodiment where a myelin antigen could be any of the following: myelin basic protein (MBP), proteolipid protein (PLP) or myelin oligodendrocyte glycoprotein (MOG). In an additional embodiment, autoantigen is a C-terminal region of MBP e.g. MBP (85-99).

In another embodiment of the present invention provides treatment for amelioration of a demyelinating disorder with at least one peptide selected from a group of peptides with amino acid sequences as set forth in SEQ ID NO: 8 to SEQ ID NO: 89 preferably SEQ ID NO: 10 to SEQ ID NO: 23; SEQ ID NO: 26 to SEQ ID NO: 34; SEQ ID NO: 39 to SEQ ID NO: 41; SEQ ID NO: 46 to SEQ ID NO: 47; SEQ ID NO: 50 to SEQ ID NO: 56; SEQ ID NO: 64 to SEQ ID NO: 68; SEQ ID NO: 86 to SEQ ID NO: 89 results in decreased frequency of myelin reactive cells in central nervous system (CNS) or peripheral lymphoid tissues.

In certain embodiment where myelin reactive cells mentioned above for example has Th1, Th17 and/or Th23 phenotype.

In another related embodiment of the present invention provides treatment for amelioration of a demyelinating disorder with at least one of the peptide selected from a group of peptides with sequences as set forth in SEQ ID NO: 8 to SEQ ID NO: 89 results into increased occurrence of peptide reactive Th2, Treg cells in CNS or peripheral lymphoid tissue.

Present invention in a major embodiment provides a therapeutic formulation comprising at least one of the following peptides having amino acid sequences as set forth in SEQ ID NO: 8 to SEQ ID NO: 89 or their homo-polymers or co-polymers for the treatment of an autoimmune, inflammatory, demyelinating condition in human subjects at a therapeutically effective dosage.

In another embodiment where therapeutic formulation mentioned above is used for the treatment of human subjects with multiple sclerosis (MS).

In yet another embodiment where therapeutic formulation is administered through any of the routes such as subcutaneous, epicutaneous, intradermal, intramuscular, intravenous, intraperitoneal, intrathecal, intracranial, oral in a pharmaceutically acceptable carrier.

In still another embodiment where above mentioned therapeutic formulation is provided in the form of a kit which contains any of the above mentioned peptides as its indispensable component. In an additionally related embodiment where the peptide mentioned above is presented in a form which could be soluble and monomeric, insoluble aggregates, oligomeric or multimeric.

In one of the embodiment as provided in the present invention, a therapeutic formulation consisting any of the peptides with amino acid sequences as set forth in SEQ ID NO: 8 to SEQ ID NO: 89, wherein any of the amino acid in the peptides is substituted by its analog, wherein substituted analog is a D-amino acid or the substituted analog is derived by addition/incorporation/modification of the parent amino acid with chemical entities/functional groups such as alkyl, formyl, phosphate, acetyl, t-butoxyl, halogens or the substituted analog is a chemical entity having similar charge and/or size properties. In another related embodiment where the β-peptide/peptide backbone is replaced with a homologous or analogous structural entity which forms an amphipathic helix and which may include replacement of one/more peptide bond with a non-peptide bond that is selected from a group consisting of —CH₂—NH—, —CH₂—S—, —CH₂—CH₂—, —CH═CH—, —CO—CH₂, —CH(OH)CH₂— and —CH₂SO—.

In yet another related embodiment where there is a substitution and/or addition of few small sized neutral amino acids and/or their analogs to C—, N— or both termini and/or penultimate positions at either or both ends, where neutral small sized amino acid could be glycine, alanine or proline.

In another embodiment of the present invention, wherein peptide selected from the group of peptides with sequences as set forth in SEQ ID NO: 8 to SEQ ID NO: 89 is modified at C—, N—, or both termini with addition of cell penetrating peptides or motifs either attached covalently, non-covalently and/or separated by linker(s) consisting of a sequence recognized and cleaved by a cell resident protease or peptidase.

In a further related embodiment of the present invention, the peptide mentioned in the previous embodiment could be HIV-1 Tat, penetratin (Antp), poly-lys, poly-arg, MPG, Pep-1, CADY, TP, TP10, transportan, VP22, model amphipathic peptide (MAP) and linker is RVKR sensitive to trans-golgi network, resident endopeptidase furin.

The present invention discloses use of β³-homoamino acids and β-amino acids in exogenous therapeutic peptides as a novel strategy to enhance their presentation cross presentation in particular by antigen presenting cells in-vivo or ex-vivo. Additionally, peptide analogs of immuno-dominant epitope of myelin basic protein (MBP), MBP (85-99) containing β³-homoamino acids or β-amino acids are being provided treatment with which effectively suppresses or ameliorates the progression of relapsing remitting (RR) or chronic progressive (CP) experimental autoimmune encephalomyelitis (EAE) in SJL/J or C57BL6/J mice by down modulating the presentation of myelin antigens.

An embodiment of the present invention provides the use of β³-homo amino acids (β-substituted-β-homo amino acids) in exogenous peptides as a strategy to enhance their presentation and/or cross presentation in particular. Further in a certain embodiment where β³-homo amino acids (β-substituted-β-homo amino acids) are replaced by their close relatives such as β-amino acids or their isomer or stereoisomer such as those having D-, L-, R-, S-configurations.

Another embodiment of the present invention provides a peptide selected from the group of peptides with sequences as set forth in SEQ ID NO: 8 to SEQ ID NO: 89, wherein the peptide is able to get localized in various cellular compartments by traversing through plasma membranes passively in a receptor independent manner and is efficiently presented and/or cross presented with class I MHC molecules on the surface of antigen presenting cells (APCs). In a certain embodiment of the present invention where the peptide mentioned above, down modulates the presentation of myelin antigens in association with class I and/or class II MHC molecules. In a further embodiment where a myelin antigen could be any of the following: myelin basic protein (MBP), proteolipid protein (PLP) or myelin oligodendrocyte glycoprotein (MOG). In an additional embodiment, autoantigen is a C-terminal region of MBP e.g. MBP (85-99).

In another embodiment of the present invention provides a peptide selected from a group of peptides with sequences as set forth in SEQ ID NO: 8 to SEQ ID NO: 89 preferably SEQ ID NO: 10 to SEQ ID NO: 23; SEQ ID NO: 26 to SEQ ID NO: 34; SEQ ID NO: 39 to SEQ ID NO: 41; SEQ ID NO: 46 to SEQ ID NO: 47; SEQ ID NO: 50 to SEQ ID NO: 56; SEQ ID NO: 64 to SEQ ID NO: 68; SEQ ID NO: 86 to SEQ ID NO: 89, wherein the peptide competes efficiently with myelin antigen derived epitopes for binding to antigen binding groove of multiple sclerosis (MS) associated class I and/or class II MHC haplotypes e.g. HLA DR2 (class II MHC) and HLA A3 (class I MHC). Further in a related embodiment where peptides mentioned above are retained for longer duration on the surface of antigen presenting cells bearing MS associated MHC haplotypes.

In another embodiment of the present invention provides a peptide selected from a group of peptides with sequences as set forth in SEQ ID NO: 8 to SEQ ID NO: 89 preferably SEQ ID NO: 10 to SEQ ID NO: 23; SEQ ID NO: 26 to SEQ ID NO: 34; SEQ ID NO: 39 to SEQ ID NO: 41; SEQ ID NO: 46 to SEQ ID NO: 47; SEQ ID NO: 50 to SEQ ID NO: 56; SEQ ID NO: 64 to SEQ ID NO: 68; SEQ ID NO: 86 to SEQ ID NO: 89, where treatment with at least one peptide results in decreased frequency of myelin reactive cells in central nervous system (CNS) or peripheral lymphoid tissues. In certain embodiment where myelin reactive cells mentioned above for example has Th1, Th17 and/or Th23 phenotype.

In another embodiment of the present invention provides a peptide selected from a group of peptides with sequences as set forth in SEQ ID NO: 8 to SEQ ID NO: 89 preferably SEQ ID NO: 10 to SEQ ID NO: 23; SEQ ID NO: 26 to SEQ ID NO: 34; SEQ ID NO: 39 to SEQ ID NO: 41; SEQ ID NO: 46 to SEQ ID NO: 47; SEQ ID NO: 50 to SEQ ID NO: 56; SEQ ID NO: 64 to SEQ ID NO: 68; SEQ ID NO: 86 to SEQ ID NO: 89, wherein treatment with at least one of the peptide results into increased occurrence of peptide reactive Th2, Treg cells in CNS or peripheral lymphoid tissue.

In another embodiment of the present invention provides a therapeutic formulation having at least one of the peptide selected from a group of peptides with sequences as set forth in SEQ ID NO: 8 to SEQ ID NO: 89 or their homo-polymers or co-polymers for the treatment of an autoimmune, inflammatory, demyelinating condition in human subjects at a therapeutically effective dosage. In another embodiment where therapeutic formulation mentioned above is used for the treatment of human subjects with multiple sclerosis (MS).

In yet another embodiment where therapeutic formulation is administered through any of the routes such as subcutaneous, epicutaneous, intradermal, intramuscular, intravenous, intraperitoneal, intrathecal, intracranial, oral in a pharmaceutically acceptable carrier. In still another embodiment where above mentioned therapeutic formulation is provided in the form of a kit which contains any of the above mentioned peptides as its indispensable component. In an additionally related embodiment where the peptide mentioned above is presented in a form which could be soluble and monomeric, insoluble aggregates, oligomeric or multimeric.

A related embodiment of the present invention provides therapeutic agent which is a random copolymer comprising key residues e.g. L-valine, L-lysine and L-tyrosine, L-alanine involved in interactions of myelin antigen derived epitopes with relevant MHCs and T-cell receptor. Further in a related embodiment where at least one of the amino acids mentioned before is a β³-homo amino acids (β-substituted-β-homo amino acids) or their close relatives i.e. β-amino acids.

In a related embodiment as provided in the present invention, a therapeutic formulation comprises any of the peptides with sequences as set forth in SEQ ID NO: 8 to SEQ ID NO: 89, where any of the amino acid in the peptides is substituted by its analog, where substituted analog is a D-amino acid or the substituted analog is derived by addition, incorporation and/or modification of the parent amino acid with chemical entities or functional groups such as alkyl, formyl, phosphate, acetyl, t-butoxyl, halogens or the substituted analog is a chemical entity having similar charge and/or size properties.

In another related embodiment where the β-peptide or peptide backbone is replaced with a homologous or analogous structural entity which forms an amphipathic helix and which may include replacement of one or more peptide bond with a non-peptide bond that is selected from a group consisting of —CH₂—NH—, —CH₂—S—, —CH₂—CH₂—, —CH═CH—, —CO—CH₂, —CH(OH)CH₂— and —CH₂SO—.

In yet another related embodiment where there is a substitution and/or addition of few small sized neutral amino acids and/or their analogs to C—, N— or both termini and/or penultimate positions at either or both ends, where neutral small sized amino acid could be glycine, alanine or proline.

In another embodiment of the present invention there is provided a peptide selected from a group of peptides with sequences as set forth in SEQ ID NO: 8 to SEQ ID NO: 89, wherein the peptide is modified at C—, N—, or both termini with addition of cell penetrating peptides or motifs either attached covalently, non-covalently and/or separated by linker(s) consisting of a sequence recognized and cleaved by a cell resident protease or peptidase.

In a further related embodiment of the present invention, the peptide mentioned in the previous embodiment could be HIV-1 Tat, penetratin (Antp), poly-lys, poly-arg, MPG, Pep-1, CADY, TP, TP10, transportan, VP22, model amphipathic peptide (MAP) and linker is RVKR sensitive to trans-golgi network, resident endopeptidase furin.

The peptide analogs as disclosed in the present invention, wherein said peptide is derived from SEQ ID NO: 1 by modification of at least one amino acid residue in SEQ ID NO: 1 to obtain a synthetic peptide having at least 5 amino acids comprising valine at position P1, tyrosine at position P4 and lysine at position P5, wherein said modification comprises substitution of at least one a amino acid by β amino acid and/or β³-homo amino acid, wherein the peptide is capable of down regulating the binding of myelin basic protein (MBP) peptide to class I and/or class II MHCs. The substitution of α amino acid by β amino acid and/or β³-homo amino acid in the said peptide results in formation of an amphipathic helix.

The substituted β³-homo amino acids or β-amino acid present in the peptide analogs disclosed in the present invention have L or D conformation with R or S stereochemistry.

The synthetic peptides as disclosed in the present invention are analogs of myelin basic protein (MBP) (85-99) (SEQ ID NO: 1) and J5 (SEQ ID NO: 4).

In addition to efficient presentation in association with class II MHC on the surface of antigen presenting cells, the peptides as disclosed in the present invention are capable to get cross presented with class I MHC (cytosolic pathway of antigen presentation), wherein antigen presenting cells mentioned are either professional or non professional antigen presenting cells for example dendritic cells, tissue specific antigen presenting cell for example are langerhan cells, microglial cells or splenic dendritic cells (SPDCs).

The peptides of the present invention are capable of blocking or inhibiting the binding of myelin antigen derived epitopes (MBP 85-99 or MBP 85-109) to class I or class II MHCs or their murine homologs which are associated with susceptibility to multiple sclerosis (MS), wherein class I MHC haplotype associated with susceptibility to MS mentioned is HLA A3 and its murine counterpart in SJL/J mice is K^{s} and class II MHC haplotype associated with susceptibility ot MS in HLA DR2 and its murine counterpart in SJL/J is I-A^{s}.

An aspect of the present invention is to provide a synthetic peptide for treatment of autoimmune and/or demyelinating conditions such as multiple sclerosis (MS), wherein the peptide comprises at least 5 amino acids and having valine at position P1, tyrosine at position P4 and lysine at position P5, wherein the peptide consists of at least one β-amino acid and/or β³-homo amino acid.

In accordance with the present invention a synthetic peptide for amelioration of a demyelinating disorder, wherein said peptide comprises at least 5 amino acids containing valine at position P1, tyrosine at position P4 and lysine at position P5. and is derived from SEQ ID NO: 1 or SEQ ID NO: 4 by modification of at least one amino acid residue, wherein the modification comprises substitution of at least one α-amino acid by β-amino acid and/or β³-homo amino acid.

In one embodiment of the present invention there is provided a synthetic peptide for amelioration of a demyelinating disorder comprising at least 5 amino acids and having valine at position PI, tyrosine at position P4 and lysine at position P5, wherein the peptide consists of at least one β-amino acid and/or β³-homo amino acid.

In another embodiment of the present invention there is provided a synthetic peptide selected from the group consisting of E K P K V E A Y K A A A A_{β}³ P_{β}³ A_{β}³ (SEQ ID NO: 10), E K P K V E A Y K A A A_{β}³ A_{β}³ P_{β}³ A_{β}³ (SEQ ID NO: 11), E K P K V E A Y K A A_{β}³ A_{β}³ A_{β}³ P_{β}³ A_{β}³ (SEQ ID NO: 12), E K P K V E A Y K A_{β}³ A_{β}³ A_{β}³ A_{β}³ P_{β}³ A_{β}³ (SEQ ID NO: 13), E K P K V E A Y K_{β}³ A_{β}³ A_{β}³ A_{β}³ A_{β}³ P_{β}³ A_{β}³ (SEQ ID NO:14), E K P K V E A Y_{β}³ K_{β}³ A_{β}³ A_{β}³ A_{β}³ A_{β}³ P_{β}³ A_{β}³ (SEQ ID NO:15), E K P K V E A_{β}³ Y_{β}³ K_{β}³ A_{β}³ A_{β}³ A_{β}³ A_{β}³ P_{β}³ A_{β}³ (SEQ ID NO: 16), E K P K V E_{β}³ A_{β}³ Y_{β}³ K_{β}³ A_{β}³ A_{β}³ A_{β}³ A_{β}³ P_{β}³ A_{β}³ (SEQ ID NO: 17), E K P K V_{β}³ E_{β}³ A_{β}³ Y_{β}³ K_{β}³ A_{β}³ A_{β}³ A_{β}³ A_{β}³ P_{β}³ A_{β}³ (SEQ ID NO: 18), E K P K_{β}³ V_{β}³ E_{β}³ A_{β}³ Y_{β}³ K_{β}³ A_{β}³ A_{β}³ A_{β}³ A_{β}³ P_{β}³ A_{β}³ (SEQ ID NO: 19), E K P_{β}³ K_{β}³ V_{β}³ E_{β}³ A_{β}³ Y_{β}³ K_{β}³ A_{β}³ A_{β}³ A_{β}³ A_{β}³ P_{β}³ A_{β}³ (SEQ ID NO: 20), E K_{β}³ P_{β}³ K_{β}³ V_{β}³ E_{β}³ A_{β}³ Y_{β}³ K_{β}³ A_{β}³ A_{β}³ A_{β}³ A_{β}³ P_{β}³ A_{β}³ (SEQ ID NO: 21), E_{β}³ K_{β}³ P_{β}³ K_{β}³ V_{β}³ E_{β}³ A_{β}³ Y_{β}³ K_{β}³ A_{β}³ A_{β}³ A_{β}³ A_{β}³ P_{β}³ A_{β}³ (SEQ ID NO: 22), E_{β}³ K_{β}³ P_{β}³ K_{β}³ V_{β}³ E A Y_{β}³ K_{β}³ A_{β}³ A_{β}³ A_{β}³ A_{β}³ P_{β}³ A_{β}³ (SEQ ID NO: 23), K V E A Y K A A_{β}³ A_{β}³ A_{β}³ (SEQ ID NO: 26), K V E A Y K A_{β}³ A_{β}³ A_{β}³ A_{β}³ (SEQ ID NO:27), K V E A Y K_{β}³ A_{β}³ A_{β}³ A_{β}³ A_{β}³ (SEQ ID NO:28), K V E A Y_{β}³ K_{β}³ A_{β}³ A_{β}³ A_{β}³ A_{β}³ (SEQ ID NO:29), K V E A_{β}³ Y_{β}³ K_{β}³ A_{β}³ A_{β}³ A_{β}³ A_{β}³ (SEQ ID NO: 30), K V E_{β}³ A_{β}³ Y_{β}³ K_{β}³ A_{β}³ A_{β}³ A_{β}³ A_{β}³ (SEQ ID NO:31), K V_{β}³ E_{β}³ A_{β}³ Y_{β}³ K_{β}³ A_{β}³ A_{β}³ A_{β}³ A_{β}³ (SEQ ID NO: 32), K_{β}³ V_{β}³ E A Y_{β}³ K_{β}³ A_{β}³ A_{β}³ A_{β}³ A_{β}³ (SEQ ID NO:33), K_{β}³ V_{β}³ E_{β}³ A_{β}³ Y_{β}³ K_{β}³ A_{β}³ A_{β}³ A_{β}³ A_{β}³ (SEQ ID NO: 34), K V_{β}³ E_{β}³ A_{β}³ Y_{β}³ K_{β}³ (SEQ ID NO:39), K_{β}³ V_{β}³ E_{β}³ A_{β}³ Y_{β}³ K_{β}³ (SEQ ID NO: 40), K_{β}³ V_{β}³ E A Y_{β}³ K_{β}³ (SEQ ID NO:41), V_{β}³ E_{β}³ A_{β}³ Y_{β}³ K_{β}³ (SEQ ID NO: 46), V_{β}³ E A Y_{β}³ K_{β}³ (SEQ ID NO:47), E K P K V E A Y K A A A_{β} A_{β} P A_{β} (SEQ ID NO: 50), E K P K V E A Y K A A_{β} A_{β} A_{β} P A_{β} (SEQ ID NO: 51), E K P K V E A Y K A_{β} A_{β} A_{β} A_{β} P A_{β} (SEQ ID NO:52), E K P K V E A Y K_{β} A_{β} A_{β} A_{β} A_{β} P A_{β} (SEQ ID NO:53), E K P K V E A Y_{β} K_{β} A_{β} A_{β} A_{β} A_{β} P A_{β} (SEQ ID NO: 54), E K P K V E A_{β} Y_{β} K_{β} A_{β} A_{β} A_{β} A_{β} P A_{β} (SEQ ID NO: 55), E K P K V E_{β} A_{β} Y_{β} K_{β} A_{β} A_{β} A_{β} A_{β} P A_{β} (SEQ ID NO: 56), K V E A Y K A A_{β} A_{β} A_{β} (SEQ ID NO:64), K V E A Y K A_{β} A_{β} A_{β} A_{β} (SEQ ID NO:65), K V E A Y K_{β} A_{β} A_{β} A_{β} A_{β} (SEQ ID NO:66), K V E A Y_{β} K_{β} A_{β} A_{β} A_{β} A_{β} (SEQ ID NO:67), K V E A_{β} Y_{β} K_{β} A_{β} A_{β} A_{β} A_{β} (SEQ ID NO:68), E_{β} K_{β} P K_{β}³ V_{β}³ E_{β}³ A_{β} Y_{β}³ K_{β}³ A_{β} A_{β} A_{β} A_{β}³ P A_{β} (SEQ ID NO:86), E_{β} K_{β} P K_{β}³ V_{β}³ E_{β}³ A_{β} Y_{β}³ K_{β}³ A_{β} A_{β} A_{β}³ A_{β}³ P A_{β} (SEQ ID NO:87), K_{β}³ V_{β}³ E_{β}³ A_{β} Y_{β}³ K_{β}³ A_{β} A_{β} A_{β} A_{β}³ (SEQ ID NO:88) and K_{β}³ V_{β}³ E_{β}³ A_{β} Y_{β}³ K_{β}³ A_{β} A_{β} A_{β}³ A_{β}³ (SEQ ID NO:89).

Another embodiment of the present invention there is provided a synthetic random copolymer of
tyrosine, glutamic acid, alanine and lysine, or
tyrosine, phenylalanine, alanine and lysine, or
tryptophan; valine, alanine and lysine
   wherein alanine is β-alanine (A_{β}) and/or β-homoalanine (A_{β}³); lysine is β-lysine (K_{β}) and/or β-homolysine (K_{β}³), tyrosine is β-tyrosine (Y_{β}) and/or β-homotyrosine (Y_{β}³); valine is β-valine (V_{β}) and/or β-homovaline (V_{β}³); glutamic acid is β-glutamic acid (E_{β}) and/or β-homoglutamic acid (E_{β}³); phenylalanine is β-phenylalanine (F_{β}) and/or β-homophenylalanine (F_{β}³) and tryptophan is β-tryptophan (W_{β}) and/or β-homotryptophan (W_{β}³).

One embodiment of the present invention provides the synthetic random copolymer as disclosed in the present invention, wherein molecular weight of the copolymer is in the range of about 5.8 to 11.5 kilodaltons.

One embodiment of the present invention provides the synthetic random copolymer as disclosed in the present invention, wherein molecular weight of the copolymer is 8.150 kilodaltons

One embodiment of the present invention provides the synthetic random copolymer comprising tyrosine, glutamic acid, alanine and lysine in the molar ratio of about 1:1.5:4.3:3.3, wherein alanine is β-alanine (A_{β}) and/or β-homoalanine (A_{β}³); lysine is β-lysine (K_{β}) and/or β-homolysine (K_{β}³), tyrosine is β-tyrosine (Y_{β}) and/or β-homotyrosine (Y_{β}³); and glutamic acid is β-glutamic acid (E_{β}) and/or β-homoglutamic acid (E_{β}³).

Another embodiment of the present invention provides the synthetic random copolymer comprising tyrosine, phenylalanine, alanine and lysine in the molar ratio of about 0.5:0.5:5:3, wherein alanine is β-alanine (A_{β}) and/or β-homoalanine (A_{β}³); lysine is β-lysine (K_{β}) and/or β-homolysine (K_{β}³), tyrosine is β-tyrosine (Y_{β}) and/or β-homotyrosine (Y_{β}³); and phenylalanine is β-phenylalanine (F_{β}) and/or β-homophenylalanine (F_{β}³).

In yet another embodiment of the present invention there is provided the synthetic random copolymer comprising tryptophan; valine, alanine and lysine in the molar ratio of about 0.5:0.5:5:3, wherein alanine is β-alanine (A_{β}) and/or β-homoalanine (A_{β}³); lysine is β-lysine (K_{β}) and/or β-homolysine (K_{β}³), valine is β-valine (V_{β}) and/or β-homovaline (V_{β}³); and tryptophan is β-tryptophan (W_{β}) and/or β-homotryptophan (W_{β}³).

The synthetic peptide or synthetic random copolymer peptide as disclosed in the present invention exhibits increased binding affinity to multiple sclerosis associated class II MHCs (HLADR2) relative to the peptide as set forth in SEQ ID NO:1, SEQ ID NO:4 or glatiramer acetate.

The synthetic peptide or synthetic random copolymer peptide as disclosed in the present invention exhibits increased binding affinity to multiple sclerosis associated class I MHCs (HLA A3) relative to the as set forth in SEQ ID NO:1, SEQ ID NO:2 or glatiramer acetate.

The synthetic peptide or synthetic random copolymer as disclosed in the present invention further comprises protecting groups at amino or carboxy terminus.

One of the embodiments of the present invention provides protecting groups at amino terminus is selected from a group consisting of benzyloxy carbonyl, t-butyloxy carbonyl, formyl, acetyl and acyl; and protecting groups at carboxy terminus is selected from a group consisting of amides, ether and esters such as benzyl, t-butyl.

The peptides and/or copolymers as disclosed in the present invention comprise amino acid having D, L, R, or S configurations.

The synthetic peptide or synthetic random copolymer as disclosed in the present invention further comprises a label selected from the group consisting of biotin, radioisotopes, enzymes, colloidal metals or fluorescent, chemiluminescent, or phosphorescent compounds.

The synthetic peptide or synthetic random copolymer as disclosed in the present invention is administered subcutaneously, epicutaneously, transdermally, intramuscularly, intravenously, intraperitoneally, intrathecally, intracranially or orally in the form of a pharmaceutically acceptable salts viz. acetates, carbonates, citrate, fumarate, lactate, phosphate, glutamate, phthalate, succinate, hydrochlorides, benzathine to a subject in need thereof.

The synthetic peptide or synthetic random copolymer as disclosed in the present invention is administered in monomeric, oligomeric or multimeric forms to a subject in need thereof.

In a further embodiment the present invention provides a composition for amelioration of a demyelinating disorder, said composition comprises one or more peptides comprising at least 5 amino acids and having valine at position P1, tyrosine at position P4 and lysine at position P5, wherein the peptide consists of at least one β-amino acid and/or β³-homo amino acid or a pharmaceutically acceptable salt thereof.

Further embodiment of the present invention provides a composition for amelioration of a demyelinating disorder, wherein said composition comprises at least one synthetic peptides selected from the group consisting of E K P K V E A Y K A A A A_{β}³ P_{β}³ A_{β}³ (SEQ ID NO: 10), E K P K V E A Y K A A A_{β}³ A_{β}³ P_{β}³ A_{β}³ (SEQ ID NO: 11), E K P K V E A Y K A A_{β}³ A_{β}³ A_{β}³ P_{β}³ A_{β}³ (SEQ ID NO: 12), E K P K V E A Y K A_{β}³ A_{β}³ A_{β}³ A_{β}³ P_{β}³ A_{β}³ (SEQ ID NO: 13), E K P K V E A Y K_{β}³ A_{β}³ A_{β}³ A_{β}³ A_{β}³ P_{β}³ A_{β}³ (SEQ ID NO:14), E K P K V E A Y_{β}³ K_{β}³ A_{β}³ A_{β}³ A_{β}³ A_{β}³ P_{β}³ A_{β}³ (SEQ ID NO:15), E K P K V E A_{β}³ Y_{β}³ K_{β}³ A_{β}³ A_{β}³ A_{β}³ A_{β}³ P_{β}³ A_{β}³ (SEQ ID NO: 16), E K P K V E_{β}³ A_{β}³ Y_{β}³ K_{β}³ A_{β}³ A_{β}³ A_{β}³ A_{β}³ P_{β}³ A_{β}³ (SEQ ID NO: 17), E K P K V_{β}³ E_{β}³ A_{β}³ Y_{β}³ K_{β}³ A_{β}³ A_{β}³ A_{β}³ A_{β}³ P_{β}³ A_{β}³ (SEQ ID NO: 18), E K P K_{β}³ V_{β}³ E_{β}³ A_{β}³ Y_{β}³ K_{β}³ A_{β}³ A_{β}³ A_{β}³ A_{β}³ P_{β}³ A_{β}³ (SEQ ID NO: 19), E K P_{β}³ K_{β}³ V_{β}³ E_{β}³ A_{β}³ Y_{β}³ K_{β}³ A_{β}³ A_{β}³ A_{β}³ A_{β}³ P_{β}³ A_{β}³ (SEQ ID NO: 20), E_{β}³ K_{β}³ P_{β}³ K_{β}³ V_{β}³ E_{β}³ A_{β}³ Y_{β}³ K_{β}³ A_{β}³ A_{β}³ A_{β}³ A_{β}³ P_{β}³ A_{β}³ (SEQ ID NO: 21), E_{β}³ K_{β}³ P_{β}³ K_{β}³ V_{β}³ E_{β}³ A_{β}³ Y_{β}³ K_{β}³ A_{β}³ A_{β}³ A_{β}³ A_{β}³ P_{β}³ A_{β}³ (SEQ ID NO: 22), E_{β}³ K_{β}³ P_{β}³ K_{β}³ V_{β}³ E A Y_{β}³ K_{β}³ A_{β}³ A_{β}³ A_{β}³ A_{β}³ P_{β}³ A_{β}³ (SEQ ID NO: 23), K V E A Y K A A_{β}³ A_{β}³ A_{β}³ (SEQ ID NO: 26), K V E A Y K A_{β}³ A_{β}³ A_{β}³ A_{β}³ (SEQ ID NO:27), K V E A Y K_{β}³ A_{β}³ A_{β}³ A_{β}³ A_{β}³ (SEQ ID NO:28), K V E A Y_{β}³ K_{β}³ A_{β}³ A_{β}³ A_{β}³ A_{β}³ (SEQ ID NO:29), K V E A_{β}³ Y_{β}³ K_{β}³ A_{β}³ A_{β}³ A_{β}³ A_{β}³ (SEQ ID NO: 30), K V E_{β}³ A_{β}³ Y_{β}³ K_{β}³ A_{β}³ A_{β}³ A_{β}³ A_{β}³ (SEQ ID NO:31), K V_{β}³ E_{β}³ A_{β}³ Y_{β}³ K_{β}³ A_{β}³ A_{β}³ A_{β}³ A_{β}³ (SEQ ID NO: 32), K_{β}³ V_{β}³ E A Y_{β}³ K_{β}³ A_{β}³ A_{β}³ A_{β}³ A_{β}³ (SEQ ID NO:33), K_{β}³ V_{β}³ E_{β}³ A_{β}³ Y_{β}³ K_{β}³ A_{β}³ A_{β}³ A_{β}³ A_{β}³ (SEQ ID NO: 34), K V_{β}³ E_{β}³ A_{β}³ Y_{β}³ K_{β}³ (SEQ ID NO:39), K_{β}³ V_{β}³ E_{β}³ A_{β}³ Y_{β}³ K_{β}³ (SEQ ID NO: 40), K_{β}³ V_{β}³ E A Y_{β}³ K_{β}³ (SEQ ID NO:41), V_{β}³ E_{β}³ A_{β}³ Y_{β}³ K_{β}³ (SEQ ID NO: 46), V_{β}³ E A Y_{β}³ K_{β}³ (SEQ ID NO:47), E K P K V E A Y K A A A_{β} A_{β} P A_{β} (SEQ ID NO: 50), E K P K V E A Y K A A_{β} A_{β} A_{β} P A_{β} (SEQ ID NO: 51), E K P K V E A Y K A_{β} A_{β} A_{β} A_{β} P A_{β} (SEQ ID NO:52), E K P K V E A Y K_{β} A_{β} A_{β} A_{β} A_{β} P A_{β} (SEQ ID NO:53), E K P K V E A Y_{β} K_{β} A_{β} A_{β} A_{β} A_{β} P A_{β} (SEQ ID NO: 54), E K P K V E A_{β} Y_{β} K_{β} A_{β} A_{β} A_{β} A_{β} P A_{β} (SEQ ID NO: 55), E K P K V E_{β} A_{β} Y_{β} K_{β} A_{β} A_{β} A_{β} A_{β} P A_{β} (SEQ ID NO: 56), K V E A Y K A A_{β} A_{β} A_{β} (SEQ ID NO:64), K V E A Y K A_{β} A_{β} A_{β} A_{β} (SEQ ID NO:65), K V E A Y K_{β} A_{β} A_{β} A_{β} A_{β} (SEQ ID NO:66), K V E A Y_{β} K_{β} A_{β} A_{β} A_{β} A_{β} (SEQ ID NO:67), K V E A_{β} Y_{β} K_{β} A_{β} A_{β} A_{β} A_{β} (SEQ ID NO:68), E_{β} K_{β} P K_{β}³ V_{β}³ E_{β}³ A_{β} Y_{β}³ K_{β}³ A_{β} A_{β} A_{β} A_{β}³ P A_{β} (SEQ ID NO:86), E_{β} K_{β} P K_{β}³ V_{β}³ E_{β}³ A_{β} Y_{β}³ K_{β}³ A_{β} A_{β} A_{β}³ A_{β}³ P A_{β} (SEQ ID NO:87), K_{β}³ V_{β}³ E_{β}³ A_{β} Y_{β}³ K_{β}³ A_{β} A_{β} A_{β} A_{β}³ (SEQ ID NO:88) and K_{β}³ V_{β}³ E_{β}³ A_{β} Y_{β}³ K_{β}³ A_{β} A_{β} A_{β}³ A_{β}³ (SEQ ID NO:89).

In a further embodiment the present invention provides a composition for amelioration of a demyelinating disorder, said composition comprises one or more synthetic random copolymer of
tyrosine, glutamic acid, alanine and lysine, or
tyrosine, phenylalanine, alanine and lysine, or
tryptophan; valine, alanine and lysine
   wherein alanine is β-alanine (A_{β}) and/or β-homoalanine (A_{β}³); lysine is β-lysine (K_{β}) and/or β-homolysine (K_{β}³), tyrosine is β-tyrosine (Y_{β}) and/or β-homotyrosine (Y_{β}³); valine is β-valine (V_{β}) and/or β-homovaline (V_{β}³); glutamic acid is β-glutamic acid (E_{β}) and/or β-homoglutamic acid (E_{β}³); phenylalanine is β-phenylalanine (F_{β}) and/or β-homophenylalanine (F_{β}³) and tryptophan is β-tryptophan (W_{β}) and/or β-homotryptophan (W_{β}³) or a pharmaceutically acceptable salt thereof.

Further embodiment of the present invention provides a composition for amelioration of a demyelinating disorder, wherein said composition comprises—

a) a plurality of the synthetic peptides comprising at least 5 amino acids and having valine at position P1, tyrosine at position P4 and lysine at position P5, wherein the peptide consists of at least one β-amino acid and/or β³-homo amino acid or a pharmaceutically acceptable salt thereof; or b) a plurality of synthetic peptides selected from the group consisting of E K P K V E A Y K A A A A_{β}³ P_{β}³ A_{β}³ (SEQ ID NO: 10), E K P K V E A Y K A A A_{β}³ A_{β}³ P_{β}³ A_{β}³ (SEQ ID NO: 11), E K P K V E A Y K A A_{β}³ A_{β}³ A_{β}³ P_{β}³ A_{β}³ (SEQ ID NO: 12), E K P K V E A Y K A_{β}³ A_{β}³ A_{β}³ A_{β}³ P_{β}³ A_{β}³ (SEQ ID NO: 13), E K P K V E A Y K_{β}³ A_{β}³ A_{β}³ A_{β}³ A_{β}³ P_{β}³ A_{β}³ (SEQ ID NO:14), E K P K V E A Y_{β}³ K_{β}³ A_{β}³ A_{β}³ A_{β}³ A_{β}³ P_{β}³ A_{β}³ (SEQ ID NO:15), E K P K V E A_{β}³ Y_{β}³ K_{β}³ A_{β}³ A_{β}³ A_{β}³ A_{β}³ P_{β}³ A_{β}³ (SEQ ID NO: 16), E K P K V E_{β}³ A_{β}³ Y_{β}³ K_{β}³ A_{β}³ A_{β}³ A_{β}³ A_{β}³ P_{β}³ A_{β}³ (SEQ ID NO: 17), E K P K V_{β}³ E_{β}³ A_{β}³ Y_{β}³ K_{β}³ A_{β}³ A_{β}³ A_{β}³ A_{β}³ P_{β}³ A_{β}³ (SEQ ID NO: 18), E K P K_{β}³ V_{β}³ E_{β}³ A_{β}³ Y_{β}³ K_{β}³ A_{β}³ A_{β}³ A_{β}³ A_{β}³ P_{β}³ A_{β}³ (SEQ ID NO: 19), E K P_{β}³ K_{β}³ V_{β}³ E_{β}³ A_{β}³ Y_{β}³ K_{β}³ A_{β}³ A_{β}³ A_{β}³ A_{β}³ P_{β}³ A_{β}³ (SEQ ID NO: 20), E K_{β}³ P_{β}³ K_{β}³ V_{β}³ E_{β}³ A_{β}³ Y_{β}³ K_{β}³ A_{β}³ A_{β}³ A_{β}³ A_{β}³ P_{β}³ A_{β}³ (SEQ ID NO: 21), E_{β}³ K_{β}³ P_{β}³ K_{β}³ V_{β}³ E_{β}³ A_{β}³ Y_{β}³ K_{β}³ A_{β}³ A_{β}³ A_{β}³ A_{β}³ P_{β}³ A_{β}³ (SEQ ID NO: 22), E_{β}³ K_{β}³ P_{β}³ K_{β}³ V_{β}³ E A Y_{β}³ K_{β}³ A_{β}³ A_{β}³ A_{β}³ A_{β}³ P_{β}³ A_{β}³ (SEQ ID NO: 23), K V E A Y K A A_{β}³ A_{β}³ A_{β}³ (SEQ ID NO: 26), K V E A Y K A_{β}³ A_{β}³ A_{β}³ A_{β}³ (SEQ ID NO:27), K V E A Y K_{β}³ A_{β}³ A_{β}³ A_{β}³ A_{β}³ (SEQ ID NO:28), K V E A Y_{β}³ K_{β}³ A_{β}³ A_{β}³ A_{β}³ A_{β}³ (SEQ ID NO:29), K V E A_{β}³ Y_{β}³ K_{β}³ A_{β}³ A_{β}³ A_{β}³ A_{β}³ (SEQ ID NO: 30), K V E_{β}³ A_{β}³ Y_{β}³ K_{β}³ A_{β}³ A_{β}³ A_{β}³ A_{β}³ (SEQ ID NO:31), K V_{β}³ E_{β}³ A_{β}³ Y_{β}³ K_{β}³ A_{β}³ A_{β}³ A_{β}³ A_{β}³ (SEQ ID NO: 32), K_{β}³ V_{β}³ E A Y_{β}³ K_{β}³ A_{β}³ A_{β}³ A_{β}³ A_{β}³ (SEQ ID NO:33), K_{β}³ V_{β}³ E_{β}³ A_{β}³ Y_{β}³ K_{β}³ A_{β}³ A_{β}³ A_{β}³ A_{β}³ (SEQ ID NO: 34), K V_{β}³ E_{β}³ A_{β}³ Y_{β}³ K_{β}³ (SEQ ID NO:39), K_{β}³ V_{β}³ E_{β}³ A_{β}³ Y_{β}³ K_{β}³ (SEQ ID NO: 40), K_{β}³ V_{β}³ E A Y_{β}³ K_{β}³ (SEQ ID NO:41), V_{β}³ E_{β}³ A_{β}³ Y_{β}³ K_{β}³ (SEQ ID NO: 46), V_{β}³ E A Y_{β}³ K_{β}³ (SEQ ID NO:47), E K P K V E A Y K A A A_{β} A_{β} P A_{β} (SEQ ID NO: 50), E K P K V E A Y K A A_{β} A_{β} A_{β} P A_{β} (SEQ ID NO: 51), E K P K V E A Y K A_{β} A_{β} A_{β} A_{β} P A_{β} (SEQ ID NO:52), E K P K V E A Y K_{β} A_{β} A_{β} A_{β} A_{β} P A_{β} (SEQ ID NO:53), E K P K V E A Y_{β} K_{β} A_{β} A_{β} A_{β} A_{β} P A_{β} (SEQ ID NO: 54), E K P K V E A_{β} Y_{β} K_{β} A_{β} A_{β} A_{β} A_{β} P A_{β} (SEQ ID NO: 55), E K P K V E_{β} A_{β} Y_{β} K_{β} A_{β} A_{β} A_{β} A_{β} P A_{β} (SEQ ID NO: 56), K V E A Y K A A_{β} A_{β} A_{β} (SEQ ID NO:64), K V E A Y K A_{β} A_{β} A_{β} A_{β} (SEQ ID NO:65), K V E A Y K_{β} A_{β} A_{β} A_{β} A_{β} (SEQ ID NO:66), K V E A Y_{β} K_{β} A_{β} A_{β} A_{β} A_{β} (SEQ ID NO:67), K V E A_{β} Y_{β} K_{β} A_{β} A_{β} A_{β} A_{β} (SEQ ID NO:68), E_{β} K_{β} P K_{β}³ V_{β}³ E_{β}³ A_{β} Y_{β}³ K_{β}³ A_{β} A_{β} A_{β} A_{β}³ P A_{β} (SEQ ID NO:86), E_{β} K_{β} P K_{β}³ V_{β}³ E_{β}³ A_{β} Y_{β}³ K_{β}³ A_{β} A_{β} A_{β}³ A_{β}³ P A_{β} (SEQ ID NO:87), K_{β}³ V_{β}³ E_{β}³ A_{β} Y_{β}³ K_{β}³ A_{β} A_{β} A_{β} A_{β}³ (SEQ ID NO:88) and K_{β}³ V_{β}³ E_{β}³ A_{β} Y_{β}³ K_{β}³ A_{β} A_{β} A_{β}³ A_{β}³ (SEQ ID NO:89); or

c) a plurality of the synthetic random copolymer of
tyrosine, glutamic acid, alanine and lysine, or
tyrosine, phenylalanine, alanine and lysine, or
tryptophan; valine, alanine and lysine
   wherein alanine is β-alanine (A_{β}) and/or β-homoalanine (A_{β}³); lysine is β-lysine (K_{β}) and/or β-homolysine (K_{β}³), tyrosine is β-tyrosine (Y_{β}) and/or β-homotyrosine (Y_{β}³); valine is β-valine (V_{β}) and/or β-homovaline (V_{β}³); glutamic acid is β-glutamic acid (E_{β}) and/or β-homoglutamic acid (E_{β}³); phenylalanine is β-phenylalanine (F_{β}) and/or β-homophenylalanine (F_{β}³) and tryptophan is β-tryptophan (W_{β}) and/or β-homotryptophan (W_{β}³) or a pharmaceutically acceptable salt thereof; or

d) a combination of (a) or (b) with (c).

Further embodiment of the present invention provides a composition for amelioration of a demyelinating disorder, wherein said composition comprises—

a) at least one synthetic peptides comprising at least 5 amino acids and having valine at position P1, tyrosine at position P4 and lysine at position P5, wherein the peptide consists of at least one β-amino acid and/or β³-homo amino acid or a pharmaceutically acceptable salt thereof; or b) at least one of the synthetic peptides selected from the group consisting of E K P K V E A Y K A A A A_{β}³ P_{β}³ A_{β}³ (SEQ ID NO: 10), E K P K V E A Y K A A A_{β}³ A_{β}³ P_{β}³ A_{β}³ (SEQ ID NO: 11), E K P K V E A Y K A A_{β}³ A_{β}³ A_{β}³ P_{β}³ A_{β}³ (SEQ ID NO: 12), E K P K V E A Y K A_{β}³ A_{β}³ A_{β}³ A_{β}³ P_{β}³ A_{β}³ (SEQ ID NO: 13), E K P K V E A Y K_{β}³ A_{β}³ A_{β}³ A_{β}³ A_{β}³ P_{β}³ A_{β}³ (SEQ ID NO:14), E K P K V E A Y_{β}³ K_{β}³ A_{β}³ A_{β}³ A_{β}³ A_{β}³ P_{β}³ A_{β}³ (SEQ ID NO:15), E K P K V E A_{β}³ Y_{β}³ K_{β}³ A_{β}³ A_{β}³ A_{β}³ A_{β}³ P_{β}³ A_{β}³ (SEQ ID NO: 16), E K P K V E_{β}³ A_{β}³ Y_{β}³ K_{β}³ A_{β}³ A_{β}³ A_{β}³ A_{β}³ P_{β}³ A_{β}³ (SEQ ID NO: 17), E K P K V_{β}³ E_{β}³ A_{β}³ Y_{β}³ K_{β}³ A_{β}³ A_{β}³ A_{β}³ A_{β}³ P_{β}³ A_{β}³ (SEQ ID NO: 18), E K P K_{β}³ V_{β}³ E_{β}³ A_{β}³ Y_{β}³ K_{β}³ A_{β}³ A_{β}³ A_{β}³ A_{β}³ P_{β}³ A_{β}³ (SEQ ID NO: 19), E K P_{β}³ K_{β}³ V_{β}³ E_{β}³ A_{β}³ Y_{β}³ K_{β}³ A_{β}³ A_{β}³ A_{β}³ A_{β}³ P_{β}³ A_{β}³ (SEQ ID NO: 20), E K_{β}³ P_{β}³ K_{β}³ V_{β}³ E_{β}³ A_{β}³ Y_{β}³ K_{β}³ A_{β}³ A_{β}³ A_{β}³ A_{β}³ P_{β}³ A_{β}³ (SEQ ID NO: 21), E_{β}³ K_{β}³ P_{β}³ K_{β}³ V_{β}³ E_{β}³ A_{β}³ Y_{β}³ K_{β}³ A_{β}³ A_{β}³ A_{β}³ A_{β}³ P_{β}³ A_{β}³ (SEQ ID NO: 22), E_{β}³ K_{β}³ P_{β}³ K_{β}³ V_{β}³ E A Y_{β}³ K_{β}³ A_{β}³ A_{β}³ A_{β}³ A_{β}³ P_{β}³ A_{β}³ (SEQ ID NO: 23), K V E A Y K A A_{β}³ A_{β}³ A_{β}³ (SEQ ID NO: 26), K V E A Y K A_{β}³ A_{β}³ A_{β}³ A_{β}³ (SEQ ID NO:27), K V E A Y K_{β}³ A_{β}³ A_{β}³ A_{β}³ A_{β}³ (SEQ ID NO:28), K V E A Y_{β}³ K_{β}³ A_{β}³ A_{β}³ A_{β}³ A_{β}³ (SEQ ID NO:29), K V E A_{β}³ Y_{β}³ K_{β}³ A_{β}³ A_{β}³ A_{β}³ A_{β}³ (SEQ ID NO: 30), K V E_{β}³ A_{β}³ Y_{β}³ K_{β}³ A_{β}³ A_{β}³ A_{β}³ A_{β}³ (SEQ ID NO:31), K V_{β}³ E_{β}³ A_{β}³ Y_{β}³ K_{β}³ A_{β}³ A_{β}³ A_{β}³ A_{β}³ (SEQ ID NO: 32), K_{β}³ V_{β}³ E A Y_{β}³ K_{β}³ A_{β}³ A_{β}³ A_{β}³ A_{β}³ (SEQ ID NO:33), K_{β}³ V_{β}³ E_{β}³ A_{β}³ Y_{β}³ K_{β}³ A_{β}³ A_{β}³ A_{β}³ A_{β}³ (SEQ ID NO: 34), K V_{β}³ E_{β}³ A_{β}³ Y_{β}³ K_{β}³ (SEQ ID NO:39), K_{β}³ V_{β}³ E_{β}³ A_{β}³ Y_{β}³ K_{β}³ (SEQ ID NO: 40), K_{β}³ V_{β}³ E A Y_{β}³ K_{β}³ (SEQ ID NO:41), V_{β}³ E_{β}³ A_{β}³ Y_{β}³ K_{β}³ (SEQ ID NO: 46), V_{β}³ E A Y_{β}³ K_{β}³ (SEQ ID NO:47), E K P K V E A Y K A A A_{β} A_{β} P A_{β} (SEQ ID NO: 50), E K P K V E A Y K A A_{β} A_{β} A_{β} P A_{β} (SEQ ID NO: 51), E K P K V E A Y K A_{β} A_{β} A_{β} A_{β} P A_{β} (SEQ ID NO:52), E K P K V E A Y K_{β} A_{β} A_{β} A_{β} A_{β} P A_{β} (SEQ ID NO:53), E K P K V E A Y_{β} K_{β} A_{β} A_{β} A_{β} A_{β} P A_{β} (SEQ ID NO: 54), E K P K V E A_{β} Y_{β} K_{β} A_{β} A_{β} A_{β} A_{β} P A_{β} (SEQ ID NO: 55), E K P K V E_{β} A_{β} Y_{β} K_{β} A_{β} A_{β} A_{β} A_{β} P A_{β} (SEQ ID NO: 56), K V E A Y K A A_{β} A_{β} A_{β} (SEQ ID NO:64), K V E A Y K A_{β} A_{β} A_{β} A_{β} (SEQ ID NO:65), K V E A Y K_{β} A_{β} A_{β} A_{β} A_{β} (SEQ ID NO:66), K V E A Y_{β} K_{β} A_{β} A_{β} A_{β} A_{β} (SEQ ID NO:67), K V E A_{β} Y_{β} K_{β} A_{β} A_{β} A_{β} A_{β} (SEQ ID NO:68), E_{β} K_{β} P K_{β}³ V_{β}³ E_{β}³ A_{β} Y_{β}³ K_{β}³ A_{β} A_{β} A_{β} A_{β}³ P A_{β} (SEQ ID NO:86), E_{β} K_{β} P K_{β}³ V_{β}³ E_{β}³ A_{β} Y_{β}³ K_{β}³ A_{β} A_{β} A_{β}³ A_{β}³ P A_{β} (SEQ ID NO:87), K_{β}³ V_{β}³ E_{β}³ A_{β} Y_{β}³ K_{β}³ A_{β} A_{β} A_{β} A_{β}³ (SEQ ID NO:88) and K_{β}³ V_{β}³ E_{β}³ A_{β} Y_{β}³ K_{β}³ A_{β} A_{β} A_{β}³ A_{β}³ (SEQ ID NO:89); or

c) at least one synthetic random copolymer of
tyrosine, glutamic acid, alanine and lysine, or
tyrosine, phenylalanine, alanine and lysine, or
tryptophan; valine, alanine and lysine
   wherein alanine is β-alanine (A_{β}) and/or β-homoalanine (A_{β}³); lysine is β-lysine (K_{β}) and/or β-homolysine (K_{β}³), tyrosine is β-tyrosine (Y_{β}) and/or β-homotyrosine (Y_{β}³); valine is β-valine (V_{β}) and/or β-homovaline (V_{β}³); glutamic acid is β-glutamic acid (E_{β}) and/or β-homoglutamic acid (E_{β}³); phenylalanine is β-phenylalanine (F_{β}) and/or β-homophenylalanine (F_{β}³) and tryptophan is β-tryptophan (W_{β}) and/or β-homotryptophan (W_{β}³) or a pharmaceutically acceptable salt thereof; or

d) a combination of (a) or (b) with (c).

Another embodiment of the present invention relates to the demyelinating disorder selected from a group consisting of multiple sclerosis (MS), optic spinal MS, Devic's disease, Acute disseminated encephalomyelitis, Balo concentric sclerosis, Schilder disease, Marburg multiple sclerosis, Guillain-Barré syndrome, chronic inflammatory demyelinating polyneuropathy, Myalgic encephalomyelitis and Experimental autoimmune encephalomyelitis.

Yet another embodiment of the present invention relates to the multiple sclerosis selected from a group consisting of relapsing remitting multiple sclerosis, secondary progressive multiple sclerosis, primary progressive multiple sclerosis and chronic progressive multiple sclerosis.

The composition comprising more that one synthetic peptide as disclosed in the present invention, wherein the synthetic peptides is joined by a linker.

Yet another embodiment of the present invention provides a kit comprising at least one synthetic peptide comprising at least 5 amino acids and having valine at position P1, tyrosine at position P4 and lysine at position P5, wherein the peptide consists of at least one β-amino acid and/or β³-homo amino acid or a pharmaceutically acceptable salt thereof.

Yet another embodiment of the present invention provides a kit comprising at least one synthetic peptide comprising at least 5 amino acids and having valine at position P1, tyrosine at position P4 and lysine at position P5, wherein the peptide consists of at least one β-amino acid and/or β³-homo amino acid or a pharmaceutically acceptable salt thereof and a synthetic random copolymer of
tyrosine, glutamic acid, alanine and lysine, or
tyrosine, phenylalanine, alanine and lysine, or
tryptophan; valine, alanine and lysine
   wherein alanine is β-alanine (A_{β}) and/or β-homoalanine (A_{β}³); lysine is β-lysine (K_{β}) and/or β-homolysine (K_{β}³), tyrosine is β-tyrosine (Y_{β}) and/or β-homotyrosine (Y_{β}³); valine is β-valine (V_{β}) and/or β-homovaline (V_{β}³); glutamic acid is β-glutamic acid (E_{β}) and/or β-homoglutamic acid (E_{β}³); phenylalanine is β-phenylalanine (F_{β}) and/or β-homophenylalanine (F_{β}³) and tryptophan is β-tryptophan (W_{β}) and/or β-homotryptophan (W_{β}³) or a pharmaceutically acceptable salt thereof.

Yet another embodiment of the present invention provides a kit comprising at least one synthetic peptide as disclosed in the present invention and at least one synthetic random copolymer as disclosed in the present invention.

Yet another embodiment of the present invention provides a kit comprising synthetic random copolymer of
tyrosine, glutamic acid, alanine and lysine, or
tyrosine, phenylalanine, alanine and lysine, or
tryptophan; valine, alanine and lysine
   wherein alanine is β-alanine (A_{β}) and/or β-homoalanine (A_{β}³); lysine is β-lysine (K_{β}) and/or β-homolysine (K_{β}³), tyrosine is β-tyrosine (Y_{β}) and/or β-homotyrosine (Y_{β}³); valine is β-valine (V_{β}) and/or β-homovaline (V_{β}³); glutamic acid is β-glutamic acid (E_{β}) and/or β-homoglutamic acid (E_{β}³); phenylalanine is β-phenylalanine (F_{β}) and/or β-homophenylalanine (F_{β}³) and tryptophan is β-tryptophan (W_{β}) and/or β-homotryptophan (W_{β}³) or a pharmaceutically acceptable salt thereof.

Another embodiment of the present invention provides a method of ameliorating a demyelinating disorder, wherein the method comprises administering to a subject in need thereof an effective amount of one or more peptides or the synthetic copolymer disclosed in the present invention alone or in combination.

Another embodiment of the present invention provides effective amount of the synthetic peptide or the random copolymer for ameliorating a demyelinating disorder, wherein the effective amount is in the range of 1X, 2X, 3X, 4X, 5X, where X is 2.5 mg/kg body weight.

Further embodiment of the present invention provides use of the peptide and the synthetic copolymers as claimed disclosed in the present invention for the preparation of medicament for amelioration of a demyelinating disorder.

The method of ameliorating a demyelinating disorder, wherein the method comprises administering to a subject in need thereof an effective amount of one or more peptides or the synthetic copolymer disclosed in the present invention alone or in combination, wherein said subject is mammal.

The method of ameliorating a demyelinating disorder, wherein the method comprises administering to a subject in need thereof an effective amount of one or more peptides or the synthetic copolymer disclosed in the present invention alone or in combination, wherein said subject is human.

### EXAMPLES

It should be understood that the examples described are for illustrative purposes only and that various modifications or changes in the light of specification suggested to the person skilled in the art and are to be included within the spirit and purview of this application and the scope of the appended claims.

### Example 1

### Design of Myelin Basic Protein (MBP) Analogs

An analog of MBP (85-99, SEQ ID NO. 1), J5 (SEQ ID NO. 4), which is known to effectively block the binding of MBP (85-99) to HLADR2 molecules immobilized on the surface of ELISA plates or on the surface of antigen presenting cells, was found to have moderate efficacy in mice with experimental autoimmune encephalomyelitis (EAE) may be due to its limited biological half life and its inefficient uptake or presentation, thus its ability to modulate presentation of myelin antigens. Since the roles of myelin specific CD4+ and CD8+ T-cells are well known in the pathophysiology of multiple sclerosis (MS) or EAE, herein J5 (SEQ ID NO. 4) was designed to have properties of β-peptide by replacing some of the α-amino acids with β³-homoamino acids or (β-amino acids in a manner that its ability to bind to major histocompatibility complex (MHC) molecules is retained.

### Synthesis and Characterization of Peptides and Random Copolymers

All the peptides used were commercially synthesized from Anaspec, Syngene, Bioconcept, and Genpro-biotech. However, the peptides can be synthesized employing standard method known in the art such as protection deprotection chemistry and can be purified known methods such as by reverse phase HPLC.

Copolymer S101 (Y_{β}³ E_{β}³ A_{β}³ K_{β}³) was prepared by polymerization of the of β-HTyr N-carboxy anhydride, β-HGlu N-carboxy anhydride, β-HAla N-carboxy anhydride and β-Lys N-carboxy anhydride employing solid phase synthesis. The polymerization was carried out using Fmoc protected N-carboxyanhydrides of respective amino acids mixed in desired ratios at each cycle. The end product is a mixture of acetate salts of random peptides. Copolymer S101 (Y_{β}³ E_{β}³ A_{β}³ K_{β}³) is obtained in the molar ratio 1:1.5:4.3:3.3 and MW_{avg}4700-11000 Da Purity >95%

Copolymer S102 (Y_{β}³ F_{β}³ A_{β}³ K_{β}³) was synthesized using the Fmoc protected β-HTyr N-carboxy anhydride, β-HPhe N-carboxy anhydride, (β-HAla N-carboxy anhydride and β-Lys N-carboxy anhydride employing solid phase chemistry. The solution of each chosen amino acid in its carboxyanhydride form was added in desired ratios at each cycle of peptide synthesis. The complete copolymer was cleaved from the resin and obtained as acetate salt (Purity >95%) with MWavg 4700-11000 Da. The molar ratio was found approximating 1:1.5:4.3:3.3.

Copolymer S103 (V_{β}³ W_{β}³ A_{β}³ K_{β}³) was synthesized using Fmoc protected β-HVal N-carboxyanhydride, β-HTrp N-carboxyanhydride, β-HAla N-carboxy anhydride and β-Lys N-carboxy anhydride using similar strategies for the above mentioned peptides. The MWavg of the copolymer was in the range 4700-11000 Da and 95% pure. Its molar ratio approximated to 1:1.5:4.3:3.3

S104 (Y_{β} E_{β} A_{β} K_{β}) the Fmoc protected N-carboxy anhydrides of β-HTyr, β-HGlu, β-HAla and β-Lys were dissolved in dioxane. The solution of each carboxyanhydride of desired amino acid was added in required ratios at each cycle of synthesis. The polymer was cleaved from resin washed and dried. Purity was 96% and molar ratio 1:1.5:4.3:3.3 with an average molecular weight 4700-11000 Da.

S105 (Y_{β} F_{β} A_{β} K_{β}) and S106 (Y_{β} W_{β} A_{β} K_{β}): The above mentioned protocol was followed with appropriate N-carboxyanhydrides of desired amino acids to synthesize these polymers. The molar ratio of these peptides was 0.5:0.5:5:3 and MW_{avg}4700-11000 Da with a purity of 95%.

S107 (Y_{β}³ E_{β}³ A_{β} K_{β}³) was prepared using the Fmoc protected β-HTyr N-carboxy anhydride, β-HGlu N-carboxy anhydride, β-HAla N-carboxy anhydride and β-Lys N-carboxy anhydride employing peptide synthesis protocols described earlier. The copolymer obtained was 95% pure with an MW_{avg}4700-11000 Da. The molar ratio of the peptide was approximately 1:1.5:4.3:3.3

S108 (Y_{β} E_{β} A_{β} K_{β}³) and S109 (V_{β}³ W_{β}³ A_{β} K_{β}³) were synthesized using Fmoc protected N-carboxyanhydrides of desired amino acids for the respective peptides. The copolymers were prepared employing similar synthetic strategy described earlier. The copolymer were cleaved from the resin, washed, dried and obtained as acetate salt (Purity >95%) with MWavg 4700-11000 Da. The molar ratio was found approximating 0.5:0.5:5:3.

Copolymer J91 (YFAK) and J92 (VWAK) were synthesized using similar protocols described for peptides mentioned earlier and were obtained in the molar ratio of 1:1.5:4.3:3.3 and their average molecular weight in an approximate range of 4700-11000 Da.

Copolymer GA (YEAK) was obtained from Natco pharma (India) in the molar ratio of 1 Y:1.5 E:4.3 A:3.3 K, with an MW_{avg}4700-11000 Da.

Details of the synthetic peptides and the synthetic peptide copolymer are provided in Table 2 and Table 3 respectively.

### Preparation of Spleen Derived Dendritic Cells

Spleen derived dendritic cells were isolated to >95% purity using plasmacytoid dendritic cell isolation kit from Miltenyi Biotech. Spleenocytes were isolated from spleen of SJL/J mice, minced and passed through a 70 micron cell strainer (BD Falcon) to get a single cell suspension. The resulting spleenocytes were first depleted of CD3+ T cells and CD 49b/pan NK+ cells using LD depletion columns provided in the kit. The CD3 and CD 49b negative cell population was collected, counted and positive selection of CD11c+ dendritic cells was performed on positive selection columns provided in the kit. The CD11c+ population was collected by removing the column from the magnetic stand, stained with trypan blue to check the viability and purity was determined using flow cytometry.

### Cell Line

HLA DR2 molecules were affinity purified from MGAR (a lymphoblastoid B cell line expressing HLA DR2 obtained from IHWG, Seattle, Wash. —USA) cell line. Cells were cultured in RPMI 1640 supplemented with 10% FBS, 2 mM glutamine, 50 U/ml penicillin and 50 μg/ml streptomycin. The anti-DR antibody L243 was purchased from Santacruz Biotech.

### Purification of HLA DR2

HLA DR2 was purified to a purity of 90-95% by immunoaffinity purification. Briefly, MGAR cells were detergent solubilized to prepare the membrane fraction which was passed sequentially through sepharose CL-6B, normal mouse serum-affinity-gel, Protein A sepharose CL-4B and L243-protein A sepharose-CL-4B at a flow rate of around 10-11 ml/h. The final eluate was dialyzed against 0.1% deoxycholate, 10 mM Tris-HCl (pH 8.0) and concentrated using centricon concentrators from Millipore. Protein concentration was determined using bicinchoninic acid (BCA) assay (Sigma). The obtained protein fraction was also run on SDS-PAGE gel to confirm identity and purity.

### Binding Assay of Peptide Analogs and Copolymers to HLA DR2

Copolymers (GA, J91, J92, S101 to S109) and peptide analogs (S1-S82) at a final concentration of 5 μM were coincubated with biotinylated MBP (85-99; purchased from Bioconcept Labs Pvt. Ltd. India) at a final concentration of 0.5 μM and HLA DR2 (0.5 μg/sample) molecules for 40 h at 37° C. and transferred to a 96-well microtiter assay plates coated with 1 μg/well purified L243 mAb. Coating of microtiter plates was performed with 100 μl of L243 mAb in PBS for 18 h at 4° C. Bound biotinylated MBP (85-99) was detected using streptavidin conjugated horse radish peroxidase (HRP). 3,3′,5,5′-tetramethylbenzidine (TMB, substrate for AP) was added to each well and absorbance at 410 nm was recorded on an ELISA reader (TECAN infinite M200).

### Mice, EAE Induction and Assessment

SJL/J and C57BL6/J mice procured from Jackson's laboratory (Bar Harbor, Me.) were maintained under standard housing conditions in the central animal facility at NII as per institutional ethical committee guidelines. 8-10 week old female SJL/J mice were used throughout the study.

To induce RR EAE or chronic progressive EAE, SJL/J or C57BL6/J mice were immunized subcutaneously with 0.2 mg of MBP (85-109) or 0.1 mg MOG (35-55) emulsified in CFA and 200 ng of pertussis toxin was injected intraperitoneally on day 0 and day 2 so as to permeablize the blood brain barrier. All the animals in various experimental groups were scored daily for clinical disability on a scale of 0-6 where 0=no neurological symptoms, 1=limp tail, 2=weakness of hind limbs or ataxia, 3=incomplete paralysis of hind limbs, 4=complete paralysis of hind limbs, 5=complete paralysis of all four limbs, 6=dead. Diseased animals were provided easy access to food and water.

### Cytokine Analysis

Levels of various cytokines in culture supernates were determined by sandwich ELISA using multiplex cytokine ELISA kit (Millipore). Briefly, 100 μl of cell culture supernates from various groups were incubated with antibody (against various pro and anti-inflammatory cytokines) coated fluorescent polystyrene beads in 96 well microtiter plates, stained with PE-conjugated secondary antibody, provided in the kit and samples were acquired on Luminometer (Bio-rad).

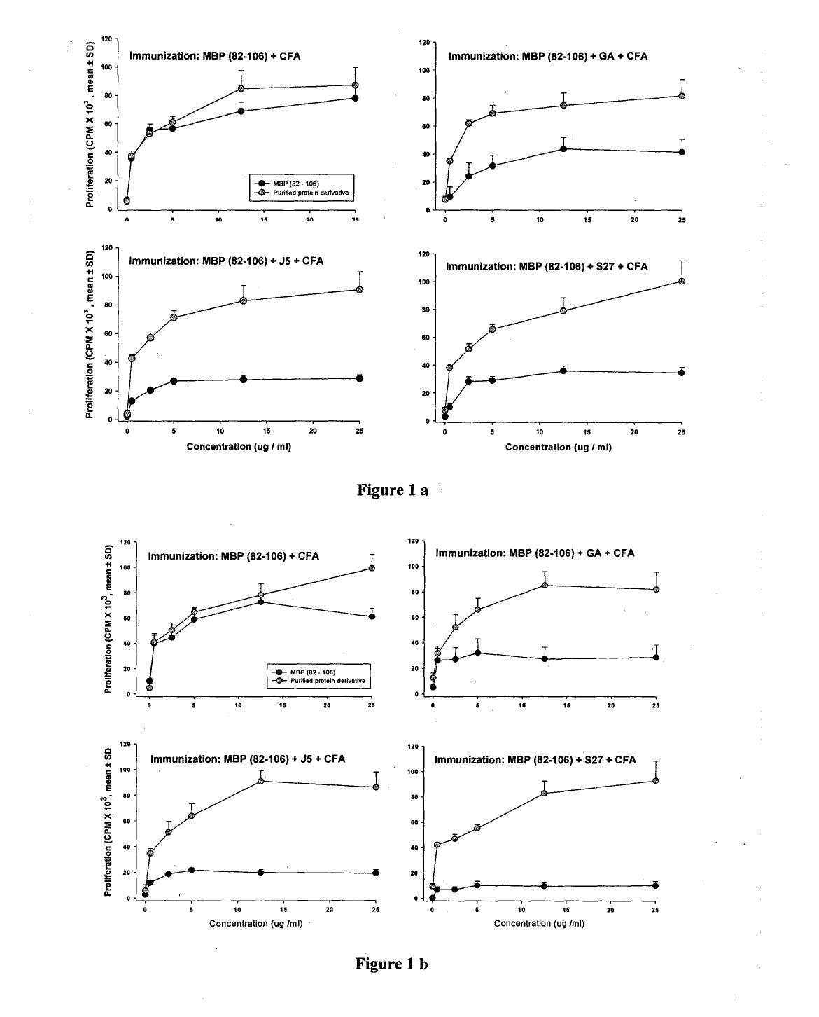

Treatment with MBP analogs designed specifically suppresses the activation of myelin reactive CD4+ T cells as proliferative in response to purified protein derivative is minimally affected (FIG. 1a). Further it specifically suppresses the activation of myelin reactive CD8+ T cells as proliferative in response to purified protein derivative is minimally affected (FIG. 1b).

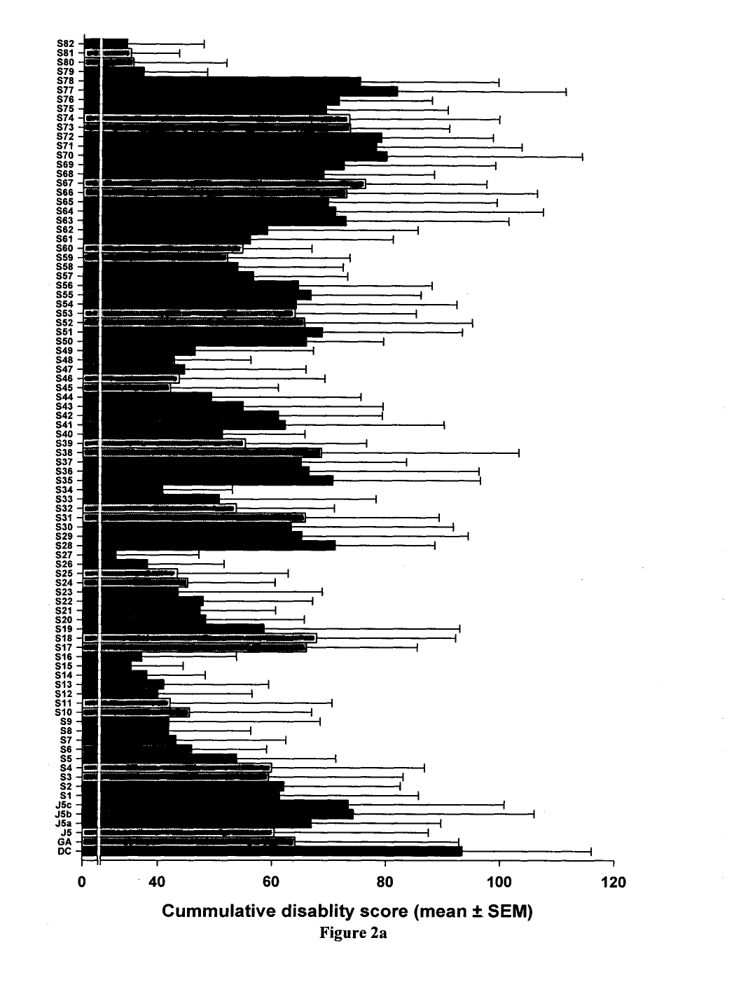

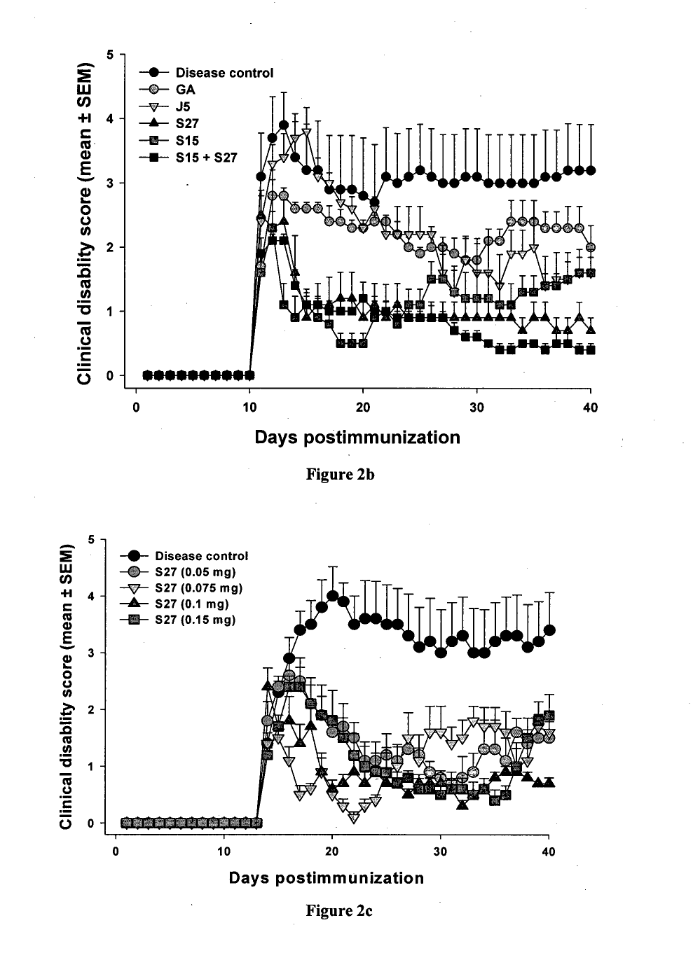

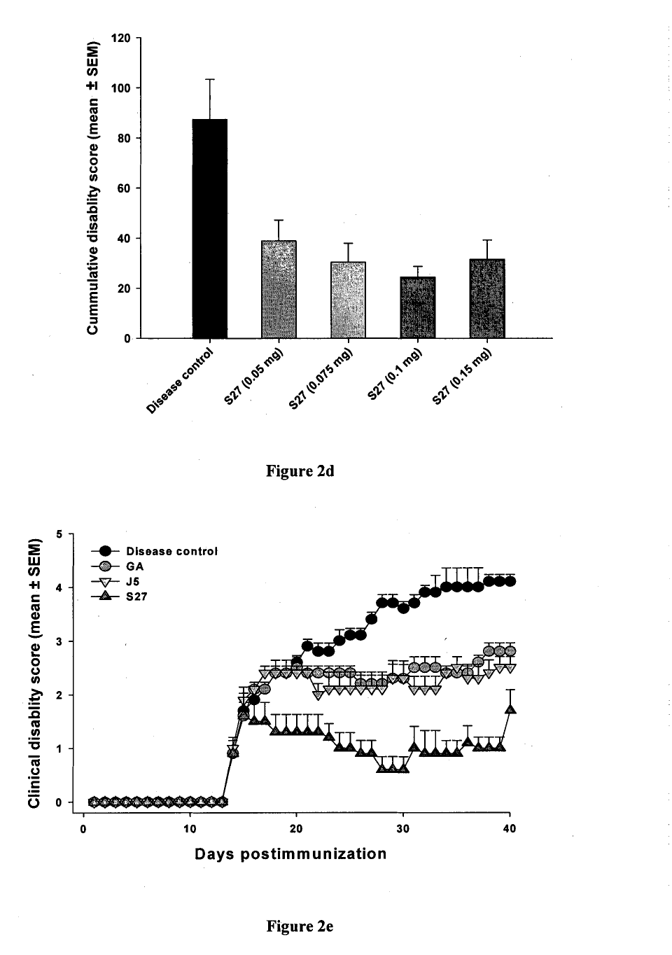

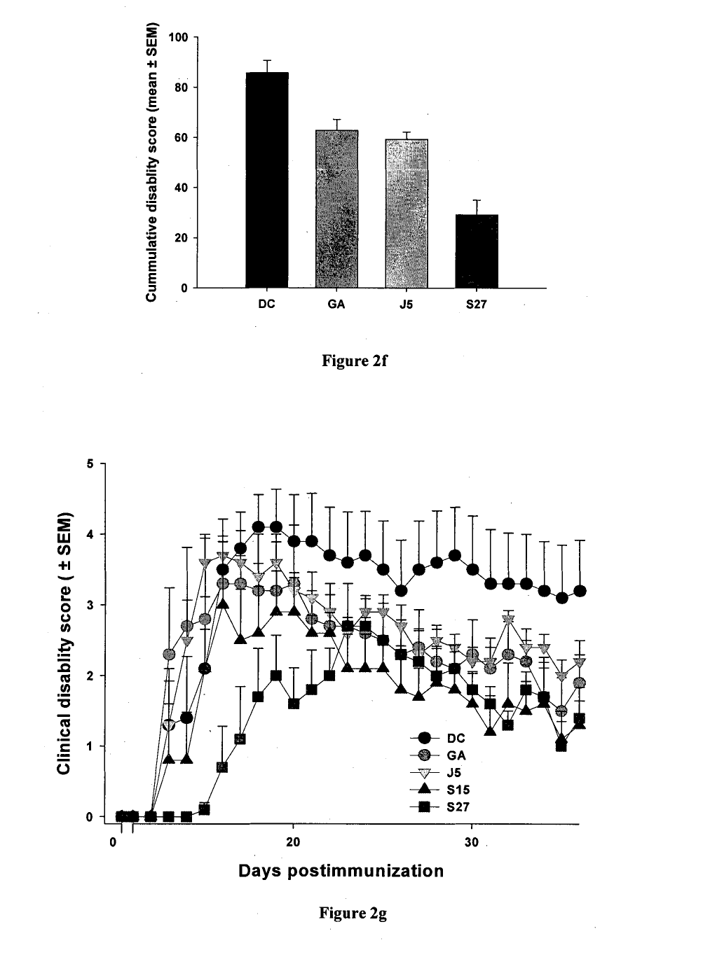

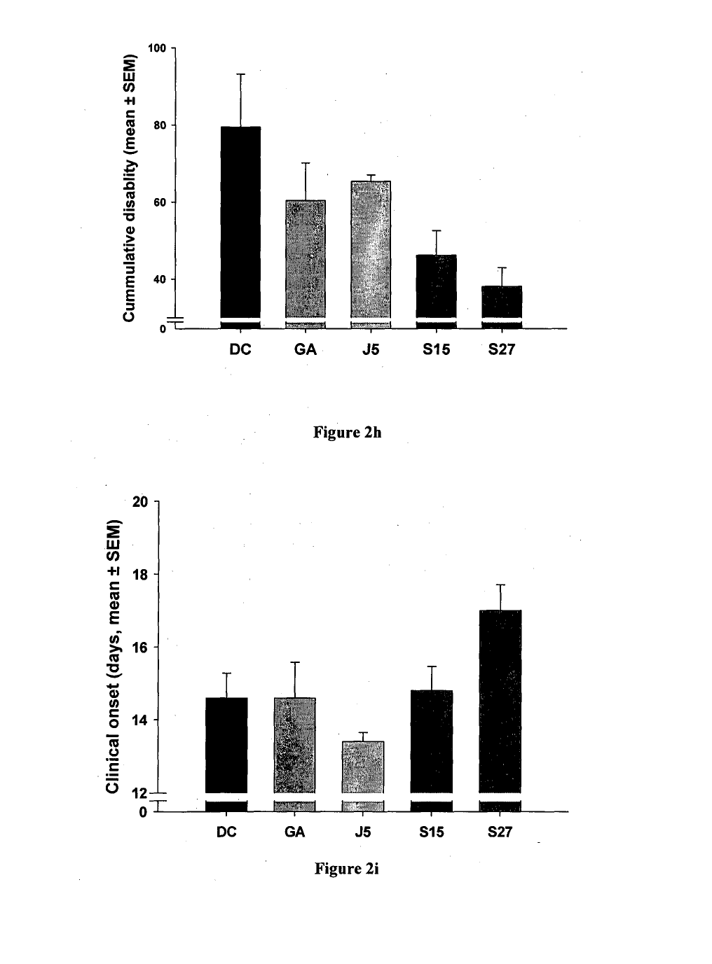

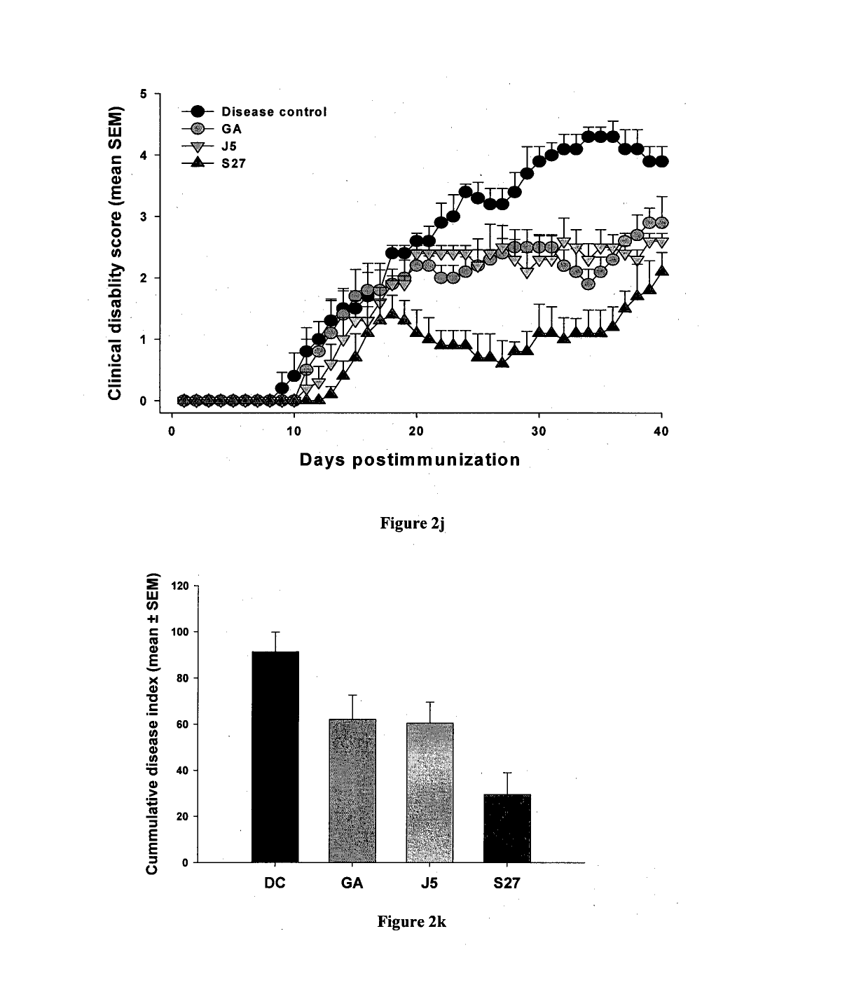

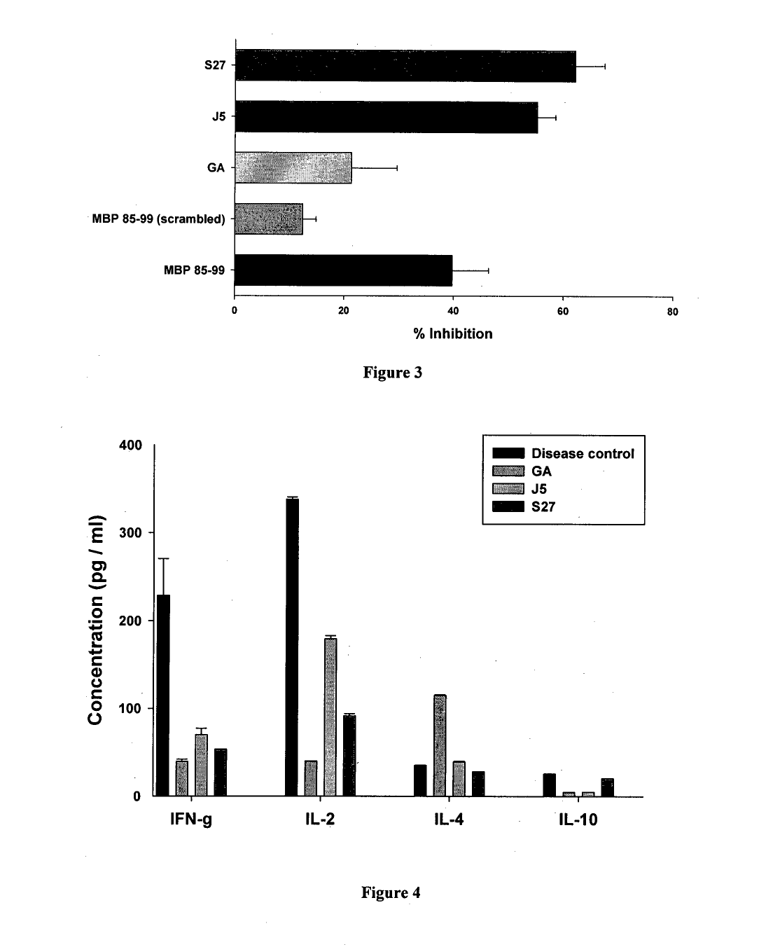

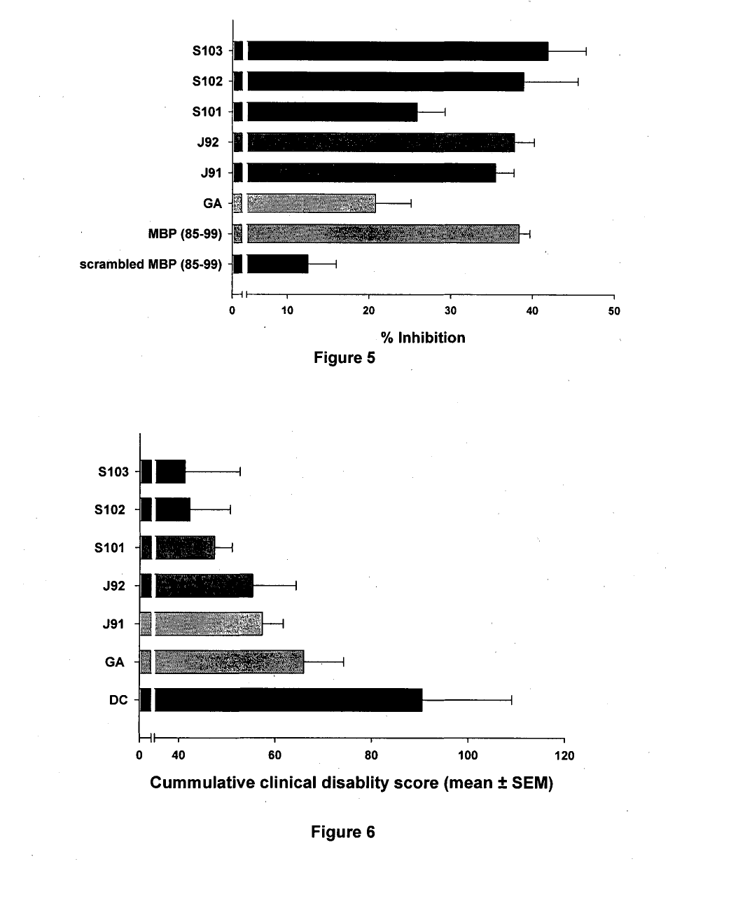

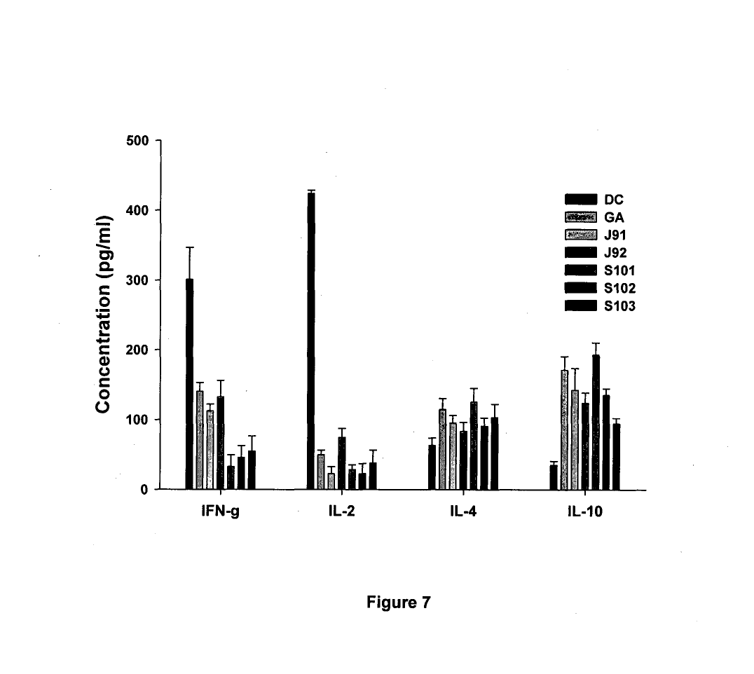

MBP analogs disclosed herein are effective for treating both relapsing remitting and chronic progressive form of multiple sclerosis (two most common disease phenotypes) FIG. 2a, b, e, f. MBP analogs designed herein are beneficial in both treatment as well as prevention (prophylactic) scenarios (FIG. 2a, b, e, f, g, h, i, j, k). Further it results into decreased levels of Th1 (proinflammatory) cytokines e.g. IFN-g and IL-2 (FIG. 3).

The peptides as disclosed in the present invention exhibit increased biological half life. The modification carried out by incorporation of β³-homoamino acids or β-amino acids into MBP (85-99) and J5 resulted in its enhanced presentation with class I and II MHC molecules, thereby, effectively down modulating presentation of myelin antigens to myelin reactive CD4+ and CD8+ T-cells. Which eventually resulted into decreased priming of myelin reactive T-cells, decreased cellular infiltration into CNS thus analogs of MBP (85-99) with β³-homoamino acids or β-amino acids are much more efficacious in the animal model of multiple sclerosis.

The peptides as disclosed in the present invention can be used in combination with any of the known therapies for example environmental enrichment, physiotherapy, acupuncture or therapeutics such as proteins or peptides e.g. IFNβ, GA, monoclonal antibodies like anti VLA4 (Tysabri), small organic molecule e.g. FTY720 (Geneliya), NBQX (inhibitor of AMPA receptor).

The peptides of the present invention specifically down-modulate the presentation of myelin antigen derived epitope e.g MBP (85-109) on the surface of antigen presenting cells with class II MHC to MBP (85-109) specific CD4+ T-cell clones in vitro or in vivo. Further the peptides analog suppresses or ameliorate the symptoms of Experimental Autoimmune Encephalomyelitis (EAE) in experimental animals or the symptoms of an autoimmune, inflammatory and/or demyelinating disorder in human subjects.

The experimental mice used in the present invention are SJL/J and C57BL6 bearing MHC haplotypes namely H-2s, H-2b respectively

The myelin antigens are derived from any of the following: myelin basic protein (MBP), Proteolipid Protein (PLP) or Myelin Oligodendrocyte Glycoprotein (MOG). The derivatives of MBP, PLP or MOG are MBP (85-109), PLP (131-151) or MOG (35-55) respectively.

### MBP Analog Containing β-Homoamino Acids/β-Amino Acids Suppresses the Progression of Relapsing Remitting (RR) and Chronic Progressive Experimental Autoimmune Encephalomyelitis (EAE)

Therapeutic activity of various MBP analogs J5 (SEQ ID NO: 4), J5a (SEQ ID NO: 5), J5b (SEQ ID NO: 6), J5c (SEQ ID NO: 7), and S1 to S 82 (SEQ ID NO: 8 to SEQ ID NO: 89), was determined in SJL/J mice exhibiting MBP (85-109) induced relapsing remitting form of EAE.

On day 11 post-immunization diseased animals displaying symptoms of neuromuscular dysfunction were grouped into various treatment groups (n=5) such that mean disability score across the groups was comparable, treated daily with vehicle or 0.1 mg of MBP analogs for two weeks and scored for clinical disability. Animals were considered diseased only when they showed definitive symptoms of EAE, e.g. complete tail paralysis, ataxia or delayed rightening reflex.

As shown in FIG. 2a majority of the MBP analogs S3 to S16, S19 to S27, S32 to S34, S39, S40, S43 to S49, S57 to S62, S79 to S82 displayed enhanced therapeutic activity in diseased animals whereas some of the analogs viz. S1, S2, S17, S18, S28 to S31, S35 to S38, S41, S42, S50 to S56, and S63 to S78 were less active than J5 for which substitutions at key contact positions (P1, P4, P5) or decreased bioavailability can be accounted. Some analogs viz. S1 to S4, S41, S42, and S62 showed therapeutic effect comparable to that of J5. Analogs namely S15, S16, S26 and S27 had maximal suppressive effect (55-65%) on clinical symptoms of the disease. Effect of GA or J5 treatment lasted only for 3-7 days in comparison to up to two weeks in case of some newly designed analogs such as S27, once the treatment is stopped, which may be attributed to their enhanced bioavailability (FIG. 2b). Dosage kinetic experiments with analog S27 representing the group of analogs comprising viz. S5 to S16, S19 to S27, S32 to S34, S39, S40, S43 to S49, S57 to S61, S79 to S82 which showed significantly improved therapeutic efficacy than J5 suggested that suppressive effect is directly proportional to the amount of peptide administered up to a certain extent and a daily dosage of 5 mg/kg body weight is optimum in the case of rodents (FIGS. 2c and 2d). In addition to MBP (85-109) induced relapsing remitting (RR) EAE in SJL/J mice which has primarily Th1 mediated etiopathology, analogs which displayed better efficacy than J5 were examined for therapeutic effects in MOG (35-55; Myelin Oligodendro Glycoprotein, a component of myelin sheath) induced chronic progressive EAE in C57BL6/J mice, a Th17 mediated disease. FIGS. 2e and 2f are depicting the therapeutic effect of analog S27, representing the group of analogs comprising analogs viz. S5 to S16, S19 to S27, S32 to S34, S39, S40, S43 to S49, S57 to S61, S79 to S82, when administered at a daily dose of 5 mg/Kg body weight. As shown in FIGS. 2e and 2f, a reduction of ˜60% in clinical disability score was observed in the case of S27.

Analogs S15 and S27 were found to suppress the progression of relapsing remitting EAE in SJL/J mice to approximately 40-50% when animals were pre-treated with 0.5 mg of S15, S27, J5 and GA in incomplete Freund's adjuvant (FIGS. 2g and 2h). Treatment with S15 or S27 also delayed the clinical onset of disease by ˜3 days whereas GA or J5 showed no effect on clinical onset of disease (FIG. 2i). Similarly an approximate 55-60% suppression was observed after pre-treatment with S27 in MOG (35-55) induced chronic EAE (FIGS. 2j and 2k). Additionally analogs namely S5 to S16, S19 to S27, S32 to S34, S39, S40, S43 to S49, S57 to S61, S79 to S82 were also found to have a suppressive effect on relapsing remitting and chronic progressive EAE when administered two weeks before immunization with MBP (85-109) or MOG (35-55). FIGS. 2g-k are highlighting the suppressive effect of S15, S27 on relapsing remitting and chronic EAE. These experiments strongly suggest that two best MBP analogs designed herein i.e. S15 and S27 are approximately two times better than the existing therapeutics i.e. GA in both treatment as well as pre-treatment scenarios. Details of the therapeutic activity of the synthetic peptides are provided in Table 4.

### Example 2

### Treatment with MBP Analogs Containing β³-Homoamino Acids/β-Amino Acids Down Modulates Recall Response to MBP (85-109)

To determine the effect of MBP analog S27 (a representative of group of analogs comparising analogs viz. S5 to S16, S19 to S27, S32 to S34, S39, S40, S43 to S49, S57 to S61, S79 to S82 which displayed superior efficacies than J5) on the priming of auto-reactive CD4+ and CD8+ T-cells by MBP (85-109), SJL/J mice were treated with 0.5 mg of S27 in incomplete Freund's Adjuvant (IFA) a day before immunization with MBP (85-109) in Freund's complete adjuvant (CFA). After two weeks spleenocytes were isolated, fractioned into CD4+ and CD8+ T-cells, cultured with MBP (85-109) pulsed spleen derived dendritic cells and assayed for proliferation (³[H]-thymidine). A considerable suppression in recall response was observed with both CD4+ and CD8+ T-cell fractions but the effect was much more pronounced in the case of CD8+ T-cell fraction when compared to J5 or GA treated group, whereas recall response to purified protein derivative (PPD, a component of mycobacterial cell wall, CFA) remained unaffected (FIGS. 1a and b). Proliferative response to PPD was measured to examine if S27 mediated suppression is specific to myelin reactive cells only whereas reactivities to other antigens remains unaffected. As recall response to MBP (85-109) is a direct measure of frequencies of MBP (85-109) reactive CD4+ or CD8+ T-cells, in the immunized animals. Thus the treatment with S27 suppresses immune response specifically to MBP (85-109) or myelin antigens.

### Example 3

### Effect of β³-Homoamino Acids/β-Amino Acids Containing MBP Analogs on the Binding of MBP (85-99) to HLA DR2

Ability of analogs viz. S1 to S82 to block the binding of immunodominant epitope MBP (85-99) to HLA DR2 was determined by incubating biotinylated MBP (85-99) with HLA DR2 in the presence of 5 μM of unlabelled MBP (85-99), GA, analogs containing β³-homoamino acids/β-amino acids or scrambled MBP (SEQ ID NO. 2). Most of the analogs designed herein viz. S1 to S82 showed inhibitory activity ranging from 15 to 60 percent. Some of the analogs e.g. S27 including J5 were infact better inhibitors than the natural ligand i.e. MBP (85-99) itself. FIG. 3 is depicting S27 to have approximately 60% inhibition in comparison to J5, GA, MBP (85-99) and scrambled MBP (85-99) having approximately 55%, 21%, 39%, 15% inhibitory activity respectively.

### Example 4

### Treatment with β³-Homoamino Acids/β-Amino Acids Containing MBP Analogs Shifts the Th1/Th2 Cytokine Balance Towards Th2

Effect of a group of analogs comprising analogs viz. S5 to S16, S19 to S27, S32 to S34, S39, S40, S43 to S49, S57 to S61, S79 to S82 represented by S27 treatment on Th1/Th2 cytokine balance was examined in culture supernates of spleenocytes isolated from various treatment groups at the end of four weeks. As depicted in FIG. 4 levels of Th1 cytokines e.g. IFN-γ and IL-2 were found to be reduced whereas that of Th2 e.g. IL-4 was found to be elevated in all the treatment groups but the effect was more pronounced in GA and S27 (SEQ ID NO: 34) treated group. Most notable effect was observed in the case of IFN-γ levels where an approximately 70% decrease was observed. Effect on Th2 cytokines was not as prominent as in the case of GA treatment group which showed ˜2 fold rise in IL-4 levels in comparison to only a marginal increase in S27 (SEQ ID NO: 34) or J5 (SEQ ID NO: 4) treatment group.

### Example 5

### Effect of β³-Homo Amino Acid Containing Copolymers on the Binding of MBP (85-99) to HLA DR2

β³-homo amino acid containing copolymers viz. S101, S102, S103, S104, S105, S106, S107, S108, S109 were synthesized and their ability to block the binding of immunodominant epitope MBP (85-99) with HLA DR2 in comparison to known copolymers such as GA, J91 and J92 was determined by incubating biotinylated MBP (85-99) with HLA DR2 in the presence of 5 μM of unlabelled MBP (85-99), GA, J91, J92, S101, S102, S103 or scrambled MBP (85-99) (SEQ ID NO: 2). Copolymers containing β³-homo amino acids viz. S101, S102, S103 were found to be better competitors than their non-β³-homo amino acid containing counterparts (GA, J91, J92) in blocking the binding of biotinylated MBP (85-99) to HLA DR2 (FIG. 5). S103 was even better than the cognate peptide MBP (85-99) at binding to HLA DR2 (FIG. 5). When compared to GA, all the β³-homo amino acid containing copolymers (S101, S102 and S103) were far superior at blocking the binding of biotinylated MBP (85-99) to HLA DR2. In addition to β³-homo amino acid containing copolymers (S101, S102 and S103), β-amino acid containing copolymers (S104, S105 and S106) and copolymers containing both β³-homo amino acid and β-amino acid (S107, S108, S109) were also examined for their ability to block the binding of MBP (85-99) to HLA DR2 and were found to have significant inhibitory activity (data not shown).

### Example 6

### Copolymers Containing β³-Homo Amino Acids Suppress the Progression of Relapsing Remitting (RR) Experimental Autoimmune Encephalomyelitis (EAE)

Therapeutic efficacy of various copolymers was determined in SJL/J mice exhibiting MBP (85-109) induced relapsing remitting form of EAE. On day 11 post immunization, diseased animals displaying symptoms of neuromuscular dysfunction were grouped into various treatment groups (n=5) such that the mean clinical disability score across the groups was comparable, treated daily with vehicle or 0.1 mg of various copolymers viz. GA, J91, J92, S101, S102, S103 for two weeks and scored for clinical disability. Animals were considered diseased only if they showed definitive symptoms of EAE, e.g. complete tail paralysis, ataxia or delayed rightening reflex. As shown in FIG. 6 and Table 5 S101, S102 and S103 were extremely effective in reducing disease severity in comparison to GA, J91 or J92. S103 displayed the maximal suppressive effect on clinical symptoms of the disease, which is about 50-55%. Details of the therapeutic activity of the synthetic peptides are provided in Table 4.

### Example 7

### Treatment with β³-Homo Amino Acid Containing Copolymers Shifts the Th1/Th2 Cytokine Balance Towards Th2

Effect of β³-homo amino acid containing copolymers viz S101, S102 and S103 on Th1/Th2 cytokine balance was studied in comparison to other copolymers viz. GA, J91, J92, for which culture supernates of spleenocytes grown in the presence of various copolymers, were assayed using ELISA to determine the levels of various cytokines. As represented in FIG. 7, the levels of Th1 cytokines e.g. IFN-γ and IL-2 were found to be significantly reduced. Most noteworthy change was in the levels of IL-2 in the S101, S102 and S103 treated groups, where the levels were brought down by 60-70%. The levels of IFN-γ were also drastically reduced by 50-60% in the S101, S102 and S103 groups respectively. Though the levels of Th2 cytokine IL-4 and IL-10 were elevated in all the treatment but the effect was most pronounced in the case of S101.

```
SEQ ID NO: 1
    E N P V V H F F K N I V T P R
SEQ ID NO: 4
    E K P K V E A Y K A A A A P A
-P4 -P3 -P2 -P1 P1 P2 P3 P4 P5 P6 P7 P8 P9 P10 P11
(Amino acid position)
```

TABLE 1
β³-homoamino acids/β-amino acids
used in various analogs or copolymers

| | Substituted | Substituted |
|---|---|---|
| Amino acid | β amino acid | β3 amino acid |
| E: L-glutamic acid | E_{β}: L-β-glutamic | E_{β}³: L-β-homoglutamic acid |
| | acid | |
| K: L-lysine | K_{β}: L-β-lysine | K_{β}³: L-β-homolysine |
| P: L-proline | NA | P_{β}³: L-β-homoproline |
| V: L-valine | V_{β}: L-β-valine | V_{β}³: L-β-homovaline |
| A: L-alanine | A_{β}: L-β-alanine | A_{β}³: L-β-homoalanine |
| Y: L-tyrosine | Y_{β}, L-β-tyrosine | Y_{β}³: L-β-homotyrosine |
| F: L-phenylalanine | F_{β}: L-β- | F_{β}³: L-β-homophenylalanine |
| | phenylalanine | |
| W: L-tryptophan | W_{β}: L-β-tryptophan | W_{β}³: L-β-homotryptophan |

Table 2
Squences of various peptides analogs

| | | Peptide | |
|---|---|---|---|
| SEQ ID NOs | Peptides | Code | Modification |
| SEQ ID NO: 1 | ENPVVHFFKNIVTPR | MBP | NO |
| | | (85-99) | |
| SEQ ID NO: 2 | FPFNVPTNIVKVERH | Scrambled | NO |
| | | MBP | |
| | | (85-99) | |
| SEQ LD NO: 3 | ENPVVHFFKNIVTPRTPPPSQGKGR | MBP | NO |
| | | (85-109) | |
| SEQ ID NO: 4 | EKPKVEAYKAAAAPA | J5 | NO |
| SEQ ID NO: 5 | KVEAYKAAAA | J5a | NO |
| SEQ ID NO: 6 | KVEAYK | J5b | NO |
| SEQ ID NO: 7 | VEAYK | J5c | NO |
| SEQ ID NO: 8 | EKPKVEAYKAAAAPA_{β}³ | S 1 | P11 |
| SEQ ID NO: 9 | EKPKVEAYKAAAAP_{β}³A_{β}³ | S 2 | P10, P11 |
| SEQ ID NO: 10 | EKPKVEAYKAAAA_{β}³P_{β}³A_{β}³ | S 3 | P9, P10, P11 |
| SEQ ID NO: 11 | EKPKVEAYKAAA_{β}³A_{β}³P_{β}³A_{β}³ | S 4 | P8, P9, P10, P11 |
| SEQ ID NO: 12 | EKPKVEAYKAA_{β}³A_{β}³A_{β}³P_{β}³A_{β}³ | S 5 | P7, P8, P9, P10, P11 |
| SEQ ID NO: 13 | EKPKVEAYK_{β}³A_{β}³A_{β}³A_{β}³A_{β}³P_{β}³A_{β}³ | S 6 | P6, P7, P8, P9, P10, P11 |
| SEQ ID NO: 14 | EKPKVEAY_{β}³K_{β}³A_{β}³A_{β}³A_{β}³A_{β}³P_{β}³A_{β}³ | S 7 | P5, P6, P7, P8, P9, P10, P11 |
| SEQ LD NO: 15 | EKPKVEAY_{β}³K_{β}³A_{β}³A_{β}³A_{β}³A_{β}³P_{β}³A_{β}³ | S 8 | P4, P5, P6, P7, P8, P9, P10, P11 |
| SEQ ID NO: 16 | EKPKVEA_{β}³Y_{β}³K_{β}³A_{β}³A_{β}³A_{β}³A_{β}³P_{β}³A_{β}³ | S 9 | P3, P4, P5, P6, P7, P8, P9, P10, P11 |
| SEQ ID NO: 17 | EKPKVE_{β}³A_{β}³Y_{β}³K_{β}³A_{β}³A_{β}³A_{β}³A_{β}³P_{β}³A_{β}³ | S 10 | P2, P3, P4, PS, P6, P7, P8, P9, P10, |
| | | | P11 |
| SEQ ID NO: 18 | EKPKV_{β}³E_{β}³A_{β}³Y_{β}³K_{β}³A_{β}³A_{β}³A_{β}³A_{β}³P_{β}³A_{β}³ | S 11 | P1, P2, P3, P4, P5, P6, P7, P8, P9, |
| | | | P10, P11 |
| SEQ ID NO: 19 | EKPK_{β}³V_{β}³E_{β}³A_{β}³Y_{β}³K_{β}³A_{β}³A_{β}³A_{β}³A_{β}³P_{β}³A_{β}³ | S 12 | -P1, P1, P2, P3, P4, P5, P6, P7, P8, |
| | | | P9, P10, P11 |
| SEQ ID NO: 20 | EKP_{β}³K_{β}³V_{β}³E_{β}³A_{β}³Y_{β}³K_{β}³A_{β}³A_{β}³A_{β}³A_{β}³P_{β}³A_{β}³ | S 13 | -P2, -P1, P1, P2, P3, P4, P5, P6, P7, |
| | | | P8, P9, P10, P11 |
| SEQ ID NO: 21 | EK_{β}³P_{β}³K_{β}³V_{β}³E_{β}³A_{β}³Y_{β}³K_{β}³A_{β}³A_{β}³A_{β}³A_{β}³P_{β}³A_{β}³ | S 14 | -P3, -P2, -P1, P1, P2, P3, P4, P5, P6, |
| | | | P7, P8, P9, P10, P11 |
| SEQ ID NO: 22 | E_{β}³K_{β}³P_{β}³K_{β}³V_{β}³E_{β}³A_{β}³Y_{β}³K_{β}³A_{β}³A_{β}³A_{β}³A_{β}³P_{β}³A_{β}³ | S 15 | -P4, -P3, -P2, -P1, P1, P2, P3, P4, |
| | | | P5, P6, P7, P8, P9, P10, P11 |
| SEQ ID NO: 23 | E_{β}³K_{β}³P_{β}³K_{β}³V_{β}³EAY_{β}³K_{β}³A_{β}³A_{β}³A_{β}³A_{β}³P_{β}³A_{β}³ | S 16 | -P4, -P3, -P2, -P1, P1, P4, P5, P6, |
| | | | P7, P8, P9, P10, P11 |
| SEQ ID NO: 24 | KVEAYKAAAA_{β}³ | S 17 | P9 |
| SEQ ID NO: 25 | KVEAYKAAA_{β}³A_{β}³ | S 18 | P8, P9 |
| SEQ ID NO: 26 | KVEAYKAA_{β}³A_{β}³A_{β}³ | S 19 | P7, P8, P9 |
| SEQ ID NO: 27 | KVEAYKA_{β}³A_{β}³A_{β}³A_{β}³ | S 20 | P6, P7, P8, P9 |
| SEQ ID NO: 28 | KVEAYK_{β}³A_{β}³A_{β}³A_{β}³A_{β}³ | S 21 | P5, P6, P7, P8, P9 |
| SEQ ID NO: 29 | KVEAY_{β}³K_{β}³A_{β}³A_{β}³A_{β}³A_{β}³ | S 22 | P4, P5, P6, P7, P8, P9 |
| SEQ ID NO: 30 | KVEA_{β}³Y_{β}³K_{β}³A_{β}³A_{β}³A_{β}³A_{β}³ | S 23 | P3, P4, P5, P6, P7, P8, P9 |
| SEQ ID NO: 31 | KVE_{β}³A_{β}³Y_{β}³K_{β}³A_{β}³A_{β}³A_{β}³A_{β}³ | S 24 | P2, P3, P4, P5, P6, P7, P8, P9 |
| SEQ ID NO: 32 | KV_{β}³E_{β}³A_{β}³Y_{β}³K_{β}³A_{β}³A_{β}³A_{β}³A_{β}³ | S 25 | P1, P2, P3, P4, P5, P6, P7, P8, P9 |
| SEQ ID NO: 33 | K_{β}³V_{β}³EAY_{β}³K_{β}³A_{β}³A_{β}³A_{β}³A_{β}³ | S 26 | -P1, P1, P4, P5, P6, P7, P8, P9 |
| SEQ ID NO: 34 | K_{β}³V_{β}³E_{β}³A_{β}³Y_{β}³K_{β}³A_{β}³A_{β}³A_{β}³A_{β}³ | S 27 | -P1, P1, P2, P3, P4, P5, P6, P7, P8, |
| | | | P9 |
| SEQ ID NO: 35 | KVEAYK_{β}³ | S 28 | P5 |
| SEQ ID NO: 36 | KVEAY_{β}³K_{β}³ | S 29 | P4, P5 |
| SEQ ID NO: 37 | KVEA_{β}³Y_{β}³K_{β}³ | S 30 | P3, P4, P5 |
| SEQ ID NO: 38 | KVE_{β}³A_{β}³Y_{β}³K_{β}³ | S 31 | P2, P3, P4, P5 |
| SEQ ID NO: 39 | KV_{β}³E_{β}³A_{β}³Y_{β}³K_{β}³ | S 32 | P1, P2, P3, P4, P5 |
| SEQ ID NO: 40 | K_{β}³V_{β}³E_{β}³A_{β}³Y_{β}³K_{β}³ | S 33 | -P1, P1, P2, P3, P4, P5 |
| SEQ ID NO: 41 | K_{β}³V_{β}³EAY_{β}³K_{β}³ | S 34 | -P1, P1, P4, P5 |
| SEQ ID NO: 42 | VEAYK_{β}³ | S 35 | P5 |
| SEQ ID NO: 43 | VEAY_{β}³K_{β}³ | S 36 | P4, P5 |
| SEQ ID NO: 44 | VEA_{β}³Y_{β}³K_{β}³ | S 37 | P3, P4, 135 |
| SEQ ID NO: 45 | VE_{β}³A_{β}³Y_{β}³K_{β}³ | S 38 | P2, P3, P4, P5 |
| SEQ ID NO: 46 | V_{β}³E_{β}³A_{β}³Y_{β}³K_{β}³ | S 39 | P1, P2, P3, P4, P5 |
| SEQ ID NO: 47 | V_{β}³EAY_{β}³K_{β}³ | S 40 | P1, P4, P5 |
| SEQ ID NO: 48 | EKPKVEAYKAAAAPA_{β} | S 41 | P11 |
| SEQ ID NO: 49 | EKPKVEAYKAAAA_{β}PA_{β} | S 42 | P9, P11 |
| SEQ LD NO: 50 | EKPKVEAYKAAA_{β}A_{β}PA_{β} | S 43 | P8, P9, P11 |
| SEQ ED NO: 51 | EKPKVEAYKAA_{β}A_{β}A_{β}PA_{β} | S 44 | P7, P8, P9, P11 |
| SEQ ID NO: 52 | EKPKVEAYKA_{β}A_{β}A_{β}A_{β}PA_{β} | S 45 | P6, P7, P8, P9, P11 |
| SEQ ID NO: 53 | EKPKVEAYK_{β}A_{β}A_{β}A_{β}A_{β}PA_{β} | S 46 | P5, P6, P7, P8, P9, P11 |
| SEQ BD NO: 54 | EKPKVEAY_{β}K_{β}A_{β}A_{β}A_{β}A_{β}PA_{β} | S 47 | P4, P5, P6, P7, P8, P9, P11 |
| SEQ ID NO: 55 | EKPKVEA_{β}Y_{β}K_{β}A_{β}A_{β}A_{β}A_{β}PA_{β} | S 48 | P3, P4, P5, P6, P7, P8, P9, P11 |
| SEQ ID NO: 56 | EKPKVE_{β}A_{β}Y_{β}K_{β}A_{β}A_{β}A_{β}A_{β}PA_{β} | S 49 | P2, P3, P4, P5, P6, P7, P8, P9, P11 |
| SEQ ID NO: 57 | EKPKV_{β}E_{β}A_{β}Y_{β}K_{β}A_{β}A_{β}A_{β}A_{β}PA_{β} | S 50 | P1, P2, P3, P4, P5, P6, P7, P8, P9, |
| | | | P11 |
| SEQ ID NO: 58 | EKPK_{β}V_{β}E_{β}A_{β}Y_{β}K_{β}A_{β}A_{β}A_{β}A_{β}PA_{β} | S 51 | -P1, P1, P2, P3, P4, P5, P6, P7, P8, |
| | | | P9, P11 |
| SEQ ID NO: 59 | EK_{β}PK_{β}V_{β}E_{β}A_{β}Y_{β}K_{β}A_{β}A_{β}A_{β}A_{β}PA_{β} | S 52 | -P3, -P1, P1, P2, P3, P4, P5, P6, P7, |
| | | | P8, P9, P11 |
| SEQ ID NO: 60 | E_{β}K_{β}PK_{β}V_{β}E_{β}A_{β}Y_{β}K_{β}A_{β}A_{β}A_{β}A_{β}PA_{β} | S 53 | -P4, -P3, -P1, P1, P2, P3, P4, P5, |
| | | | P6, P7, P8, P9, P11 |
| SEQ ID NO: 61 | E_{β}K_{β}PK_{β}V_{β}EAY_{β}K_{β}A_{β}A_{β}A_{β}A_{β}PA_{β} | S 54 | -P4, -P3, -P1, P1, P4, P5, P6, P7, |
| | | | P8, P9, P11 |
| SEQ ID NO: 62 | KVEAYKAAAA_{β} | S 55 | P9 |
| SEQ ID NO: 63 | KVEAYKAAA_{β}A_{β} | S 56 | P8, P9 |
| SEQ rD NO: 64 | KVEAYKAA_{β}A_{β}A_{β} | S 57 | P7, P8, P9 |
| SEQ ID NO: 65 | KVEAYKA_{β}A_{β}A_{β}A_{β} | S 58 | P6, P7, P8, P9 |
| SEQ ID NO: 66 | KVEAYK_{β}A_{β}A_{β}A_{β}A_{β} | S 59 | P5, P6, P7, P8, P9 |
| SEQ UD NO: 67 | KVEAY_{β}K_{β}A_{β}A_{β}A_{β}A_{β} | S 60 | P4, P5, P6, P7, P8, P9 |
| SEQ ID NO: 68 | KVEA_{β}Y_{β}K_{β}A_{β}A_{β}A_{β}A_{β} | S 61 | P3, P4, P5, P6, P7, P8, P9 |
| SEQ ID NO: 69 | KVE_{β}A_{β}Y_{β}K_{β}A_{β}A_{β}A_{β}A_{β} | S 62 | P2, P3, P4, P5, P6, P7, P8, P9 |
| SEQ ID NO: 70 | KV_{β}E_{β}A_{β}Y_{β}K_{β}A_{β}A_{β}A_{β}A_{β} | S 63 | P1, P2, P3, P4, P5, P6, P7, P8, P9 |
| SEQ ID NO: 71 | K_{β}V_{β}E_{β}A_{β}Y_{β}K_{β}A_{β}A_{β}A_{β}A_{β} | S 64 | -P1, P1, P2, P3, P4, P5, P6, P7, P8, |
| | | | P9 |
| SEQ ID NO: 72 | K_{β}V_{β}EAY_{β}K_{β}A_{β}A_{β}A_{β}A_{β} | S 65 | -P1, P1, P4, P5, P6, P7, P8, P9 |
| SEQ ID NO: 73 | KVEAYK_{β} | S 66 | P5 |
| SEQ ID NO: 74 | KVEAY_{β}K_{β} | S 67 | P4, P5 |
| SEQ ID NO: 75 | KVEA_{β}Y_{β}K_{β} | S 68 | P3, P4, P5 |
| SEQ ID NO: 76 | KVE_{β}A_{β}Y_{β}K_{β} | S 69 | P2, P3, P4, P5 |
| SEQ ID NO: 77 | KV_{β}E_{β}A_{β}Y_{β}K_{β} | S 70 | P1, P2, P3, P4, P5 |
| SEQ ID NO: 78 | K_{β}V_{β}E_{β}A_{β}Y_{β}K_{β} | S 71 | -P1, P1, P2, P3, P4, P5 |
| SEQ ID NO: 79 | K_{β}V_{β}EAY_{β}K_{β} | S 72 | -P1, P1, P4, P5 |
| SEQ ID NO: 80 | VEAYK_{β} | S 73 | P5 |
| SEQ ID NO: 81 | VEAY_{β}K_{β} | S 74 | P4, P5 |
| SEQ ID NO: 82 | VEA_{β}Y_{β}K_{β} | S 75 | P3, P4, P5 |
| SEQ ID NO: 83 | VE_{β}A_{β}Y_{β}K_{β} | S 76 | P2, P3, P4, P5 |
| SEQ ID NO: 84 | V_{β}E_{β}A_{β}Y_{β}K_{β} | S 77 | P1, P2, P3, P4, P5 |
| SEQ ID NO: 85 | V_{β}EAY_{β}K_{β} | S 78 | P1, P4, P5 |
| SEQ ID NO: 86 | E_{β}K_{β}PK_{β}³V_{β}³E_{β}³A_{β}Y_{β}³K_{β}³A_{β}A_{β}A_{β}A_{β}³PA_{β} | S 79 | -P4, -P3, -P1, P1, P2, P3, P4, P5,P6, |
| | | | P7, P8, P9, P11 |
| SEQ ID NO: 87 | E_{β}K_{β}PK_{β}³V_{β}³E_{β}³A_{β}Y_{β}³K_{β}³A_{β}A_{β}A_{β}³A_{β}³PA_{β} | S 80 | -P4, -P3, -P1, P1, P2, P3, P4, P5, P6, |
| | | | P7, P8, P9, P11 |
| SEQ ID NO: 88 | K_{β}³V_{β}³E_{β}³A_{β}Y_{β}³K_{β}³A_{β}A_{β}A_{β}A_{β}³ | S 81 | -P1, P1, P2, P3, P4, P5, P6, P7, P8, |
| | | | P9 |
| SEQ ID NO: 89 | K_{β}³V_{β}³E_{β}³A_{β}Y_{β}³K_{β}³A_{β}A_{β}A_{β}³A_{β}³ | S 82 | -P1, P1, P2, P3, P4, P5, P6, P7, P8, |
| | | | P9 |

TABLE 3
Various copolymers and their composition

| | Amino | | Molecular |
|---|---|---|---|
| Copolymers | acid composition | Molar ratio | weight (average) kDa |
| GA | Y E A K | 1:1.5:4.3:3.3 | (5.8-11.5) 8.150 kDa |
| J91 | Y F A K | 0.5:0.5:5:3 | (5.8-11.5) 8.150 kDa |
| J92 | V W A K | 0.5:0.5:5:3 | (5.8-11.5) 8.150 kDa |
| S101 | Y_{β}³ E_{β}³ A_{β}³ K_{β}³ | 1:1.5:4.3:3.3 | (5.8-11.5) 8.150 kDa |
| S102 | Y_{β}³ F_{β}³ A_{β}³ K_{β}³ | 0.5:0.5:5:3 | (5.8-11.5) 8.150 kDa |
| S103 | V_{β}³ W_{β}³ A_{β}³ K_{β}³ | 0.5:0.5:5:3 | (5.8-11.5) 8.150 kDa |
| S104 | Y_{β} E_{β} A_{β} K_{β} | 1:1.5:4.3:3.3 | (5.8-11.5) 8.150 kDa |
| S105 | Y_{β} F_{β} A_{β} K_{β} | 0.5:0.5:5:3 | (5.8-11.5) 8.150 kDa |
| S106 | V_{β} W_{β} A_{β} K_{β} | 0.5:0.5:5:3 | (5.8-11.5) 8.150 kDa |
| S107 | Y_{β}³ E_{β}³ A_{β} K_{β}³ | 1:1.5:4.3:3.3 | (5.8-11.5) 8.150 kDa |
| S108 | Y_{β}³ F_{β}³ A_{β} K_{β}³ | 0.5:0.5:5:3 | (5.8-11.5) 8.150 kDa |
| S109 | V_{β}³ W_{β}³ A_{β} K_{β}³ | 0.5:0.5:5:3 | (5.8-11.5) 8.150 kDa |

TABLE 4
Therapeutic activity of synthetic peptides viz.
S1 to S82 in comparison to J5 and GA

| | Therapeutic | Therapeutic activity in | Therapeutic activity in |
|---|---|---|---|
| Analogs | activity* | comparison to J5** | comparison to GA** |
| DC | 0 | −35.2941 | −31.3369 |
| GA | 31.3369 | −3.957218 | 0 |
| J5 | 35.29412 | 0 | 3.957218 |
| J5a | 28.34225 | −6.95187 | −2.99465 |
| J5b | 20.42781 | −14.8663 | −10.9091 |
| J5c | 21.39037 | −13.9037 | −9.94653 |
| S1 | 34.33155 | −0.96257 | 2.994651 |
| S2 | 33.47594 | −1.81818 | 2.139036 |
| S3 | 36.36364 | 1.069516 | 5.026736 |
| S4 | 35.82888 | 0.534757 | 4.491977 |
| S5 | 42.35294 | 7.058821176 | 11.01604118 |
| S6 | 50.80214 | 15.50801904 | 19.46523904 |
| S7 | 53.79679 | 18.50267144 | 22.45989144 |
| S8 | 55.18717 | 19.89304578 | 23.85026578 |
| S9 | 55.08021 | 19.7860939 | 23.7433139 |
| S10 | 51.22995 | 15.93582652 | 19.89304652 |
| S11 | 54.86631 | 19.57219016 | 23.52941016 |
| S12 | 57.21925 | 21.92513134 | 25.88235134 |
| S13 | 56.04278 | 20.74866075 | 24.70588075 |
| S14 | 59.25134 | 23.9572169 | 27.9144369 |
| S15 | 62.24599 | 26.9518693 | 30.9090893 |
| S16 | 60.2139 | 24.91978374 | 28.87700374 |
| S17 | 29.30481 | −5.98931 | −2.03209 |
| S18 | 27.37968 | −7.91444 | −3.95722 |
| S19 | 37.3262 | 2.032083 | 5.989303209 |
| S20 | 48.23529 | 12.94117412 | 16.89839412 |
| S21 | 49.30481 | 14.01069283 | 17.96791283 |
| S22 | 48.77005 | 13.47593348 | 17.43315348 |
| S23 | 53.47594 | 18.18181583 | 22.13903583 |
| S24 | 51.65775 | 16.36363401 | 20.32085401 |
| S25 | 53.58289 | 18.2887677 | 22.2459877 |
| S26 | 59.25134 | 23.9572169 | 27.9144369 |
| S27 | 65.13369 | 29.83956984 | 33.79678984 |
| S28 | 23.95722 | −11.3369 | −7.37968 |
| S29 | 30.26738 | −5.02674 | −1.06952 |
| S30 | 32.19251 | −3.10161 | 0.855613 |
| S31 | 29.51872 | −5.7754 | −1.81818 |
| S32 | 42.56684 | 7.27272492 | 11.22994492 |
| S33 | 45.7754 | 10.48128107 | 14.43850107 |
| S34 | 56.36364 | 21.06951636 | 25.02673636 |
| S35 | 24.38503 | −10.9091 | −6.95187 |
| S36 | 28.87701 | −6.41711 | −2.45989 |
| S37 | 30.37433 | −4.91979 | −0.96257 |
| S38 | 26.52406 | −8.77006 | −4.81284 |
| S39 | 40.85561 | 5.561494973 | 9.518714973 |
| S40 | 45.24064 | 9.946521711 | 13.90374171 |
| S41 | 33.36898 | −1.92514 | 2.032084 |
| S42 | 34.65241 | −0.64171 | 3.315506 |
| S43 | 41.28342 | 5.98930246 | 9.94652246 |
| S44 | 47.27273 | 11.97860727 | 15.93582727 |
| S45 | 54.86631 | 19.57219016 | 23.52941016 |
| S46 | 53.26203 | 17.96791209 | 21.92513209 |
| S47 | 52.29947 | 17.00534524 | 20.96256524 |
| S48 | 54.2246 | 18.93047893 | 22.88769893 |
| S49 | 50.26738 | 14.97325968 | 18.93047968 |
| S50 | 29.41176 | −5.88236 | −1.92514 |
| S51 | 26.41711 | −8.87701 | −4.91979 |
| S52 | 29.73262 | −5.5615 | −1.60428 |
| S53 | 31.5508 | −3.74332 | 0.213902 |
| S54 | 31.3369 | −3.95722 | −1.6E−06 |
| S55 | 28.55615 | −6.73797 | −2.78075 |
| S56 | 30.90909 | −4.38503 | −0.42781 |
| S57 | 39.35829 | 4.064169 | 8.02138877 |
| S58 | 42.35294 | 7.058821176 | 11.01604118 |
| S59 | 44.27807 | 8.983954866 | 12.94117487 |
| S60 | 41.39037 | 6.096254332 | 10.05347433 |
| S61 | 40 | 4.70588 | 8.6631 |
| S62 | 36.79144 | 1.497324 | 5.45454385 |
| S63 | 22.03209 | −13.262 | −9.30481 |
| S64 | 23.95722 | −11.3369 | −7.37968 |
| S65 | 25.34759 | −9.94653 | −5.98931 |
| S66 | 21.92513 | −13.369 | −9.41177 |
| S67 | 18.39572 | −16.8984 | −12.9412 |
| S68 | 26.20321 | −9.09091 | −5.13369 |
| S69 | 22.45989 | −12.8342 | −8.87701 |
| S70 | 14.4385 | −20.8556 | −16.8984 |
| S71 | 16.38503 | −18.9091 | −14.9519 |
| S72 | 15.40107 | −19.8931 | −15.9358 |
| S73 | 21.28342 | −14.0107 | −10.0535 |
| S74 | 21.39037 | −13.9037 | −9.94653 |
| S75 | 25.7754 | −9.51872 | −5.5615 |
| S76 | 23.42246 | −11.8717 | −7.91444 |
| S77 | 12.51337 | −22.7808 | −18.8235 |
| S78 | 19.43316 | −15.861 | −11.9037 |
| S79 | 60.10695 | 24.81283187 | 28.77005187 |
| S80 | 62.03209 | 26.73796556 | 30.69518556 |
| S81 | 62.45989 | 27.16577305 | 31.12299305 |
| S82 | 63.20856 | 27.91443615 | 31.87165615 |

*Therapeutic activity = % reduction in cumulative clinical disability score = {cumulative clinical disability score (Analogs) − cumulative clinical disability score (DC)} × 100/cumulative clinical disability score (DC)
**Therapeutic activity in comparison to J5 (Ψ) or GA = Therapeutic activity (Analogs) − Therapeutic activity (J5) or GA

TABLE 5
Therapeutic activity of various amino acid copolymers
viz. GA, J91, J92, S101, S102 and S103

| Copolymers | *Therapeutic activity |
|---|---|
| DC | 0 |
| GA | 27.11656 |
| J91 | 36.64861 |
| J92 | 38.88902 |
| S101 | 47.72651 |
| S102 | 53.3372 |
| S103 | 54.45702 |

*Therapeutic activity = % reduction in cumulative clinical disability score = {cumulative clinical disability score (Analogs) − cumulative clinical disability score (DC)} × 100/cumulative clinical disability score (DC)

1. A synthetic peptide for amelioration of a demyelinating disorder comprising at least 5 amino acids with valine at position PI, tyrosine at position P4 and lysine at position P5, wherein the peptide consists of at least one β-amino acid and/or β³-homo amino acid.

2. The peptide as claimed in claim 1 is selected from the group consisting of E K P K V E A Y K A A A Ap³ Pp³ Aₚ³ (SEQ ID NO: 10), E K P K V E A Y K A A Aₚ³ Aₚ³ Pₚ³ Ap³ (SEQ ID NO: 11), E K P K V E A Y K A Ap³ Ap³ A_{β}³ P_{β}³ Aₚ³ (SEQ ID NO: 12), E K P K V E A Y K Ap³ Ap³ Ap³ Aₚ³ Pₚ³ Aₚ³ (SEQ ID NO: 13), E K P K V E A Y Kₚ³ Ap³ Ap³ Ap³ Ap³ Pp³ Ap³ (SEQ ID NO:14), E K P K V E A Yₚ³ Kₚ³ Aₚ³ A_{β}³ A_{β}³ Aₚ³ P_{β}³ Aₚ³ (SEQ ID NO:15), E K P K V E Ap³ Yₚ³ Kₚ³ Aₚ³ Ap³ Aₚ³ Aₚ³ Pₚ³ Aₚ³ (SEQ ID NO: 16), E K P K V Ep³ Ap³ Yp³ Kp³ Aₚ³ Ap³ Aₚ³ Aₚ³ Pₚ³ Aₚ³ (SEQ ID NO: 17), E K P K Vₚ³ Ep³ Ap³ Yp³ Kp³ Ap³ Ap³ Ap³ Ap³ Pp³ Aₚ³ (SEQ ID NO: 18), E K P Kₚ³ Vₚ³ Eₚ³ Aₚ³ Yₚ³ Kp³ Ap³ Aₚ³ Aₚ³ Ap³ Pp³ Aₚ³ (SEQ ID NO: 19), E K Pₚ³ Kₚ³ Vₚ³ Ep³ Aₚ³ Yₚ³ Kₚ³ Aₚ³ Aₚ³ Ap³ Ap³ Pp³ Ap³ (SEQ ID NO: 20), E Kₚ³ Pₚ³ Kₚ³ Vₚ³ Eₚ³ Aₚ³ Yₚ³ Kₚ³ Ap³ Aₚ³ Aₚ³ Aₚ³ Pp³ Ap³ (SEQ ID NO: 21), Eₚ³ Kp³ Pₚ³ Kₚ³ Vₚ³ Eₚ³ Aₚ³ Yₚ³ Kₚ³ Aₚ³ Aₚ³ Aₚ³ Aₚ³ Pₚ³ Aₚ³ (SEQ ID NO: 22), Eₚ³ Kₚ³ Pₚ³ Kₚ³ Vₚ³ E A Y_{β}³ K_{β}³ A_{β}³ A_{β}³ A_{β}³ A_{β}³ P_{β}³ A_{β}³ (SEQ ID NO: 23), K V E A Y K A Ap³ A_{β}³ A_{β}³ (SEQ ID NO: 26), K V E A Y K A_{β}³ Aₚ³ A_{β}³ Ap³ (SEQ ID NO:27), K V E A Y Kₚ³ A_{β}³ Aₚ³ A_{β}³ A_{β}³ (SEQ ID NO:28), K V E A Y_{β}³ K_{β}³ Ap³ Ap³ Ap³ Ap³ (SEQ ID NO:29), K V E Aₚ³ Yₚ³ Kₚ³ Aₚ³ Aₚ³ Aₚ³ Aₚ³ (SEQ ID NO: 30), K V Ep³ Aₚ³ Yₚ³ Kₚ³ Aₚ³ Aₚ³ Aₚ³ Aₚ³ (SEQ ID NO:31), K Vₚ³ Eₚ³ Aₚ³ Yₚ³ Kₚ³ Ap³ Ap³ Ap³ Ap³ (SEQ ID NO: 32), Kₚ³ Vₚ³ E A Yₚ³ Kₚ³ Aₚ³ Aₚ³ A_{β}³ A_{β}³ (SEQ ID NO:33), Kp³ ν_{β}³ Eₚ³ Aₚ³ Yₚ³ Kₚ³ Aₚ³ Aₚ³ Aₚ³ Aₚ³ (SEQ ID NO: 34), K Vₚ³ Ep³ Ap³ Yₚ³ Kₚ³ (SEQ ID NO:39), Kₚ³ Vₚ³ Eₚ³ Ap³ Yₚ³ Kₚ³ (SEQ ID NO: 40), Kₚ³ Vₚ³ E A Yₚ³ Kₚ³ (SEQ ID NO:41), Vₚ³ E_{β}³ A_{β}³ Yₚ³ Kₚ³ (SEQ ID NO: 46), Vₚ³ E A Y_{β}³ K_{β}³ (SEQ ID NO:47), E K P K V E A Y K A A Ap Ap P Ap (SEQ ID NO: 50), E K P K V E A Y K A A_{β} A_{β} Ap P Ap (SEQ ID NO: 51), E K P K V E A Y K A_{β} Aₚ Aₚ Ap P Aₚ (SEQ ID NO:52), E K P K V E A Y Kp Ap Ap Ap Ap P Ap (SEQ ID NO:53), E K P K V E A Yp Kp Ap Ap Ap Ap P Ap (SEQ ID NO: 54), E K P K V E Aₚ Yₚ Kₚ A_{β} A_{β} A_{β} A_{β} P A_{β} (SEQ ID NO: 55), E K P K V E_{β} A_{β} Y_{β} K_{β} A_{β} Aₚ Aₚ A_{β} P A_{β} (SEQ ID NO: 56), K V E A Y K A Ap A_{β} Ap (SEQ ID NO:64), K V E A Y K Aₚ Aₚ A_{β} A_{β} (SEQ ID NO:65), K V E A Y K_{β} A_{β} A_{β} Ap Ap (SEQ ID NO:66), K V E A Y_{β} K_{β} A_{β} A_{β} A_{β} A_{β} (SEQ ID NO:67), K V E A_{β} Y_{β} K_{β} A_{β} A_{β} A_{β} A_{β} (SEQ ID NO:68), E_{β} K_{β} P K_{β}³ Vp³ E_{β}³ A_{β} Y_{β}³ K_{β}³ Ap A_{β} Ap Ap³ P Aₚ (SEQ ID NO:86), E_{β} Kₚ P Kₚ³ Vₚ³ E_{β}³ A_{β} Yₚ³ Kₚ³ Aₚ Aₚ Aₚ³ Aₚ³ P Ap (SEQ ID NO:87), Kₚ³ Vₚ³ Eₚ³ Aₚ Yₚ³ ₚ³ Aₚ Aₚ Aₚ Aₚ³ (SEQ ID NO:88) and Kₚ³ Vp³ Ep³ Aₚ Yₚ³ Kp³ Aₚ Aₚ Ap³ Aₚ³ (SEQ ID NO:89).

3. A synthetic random copolymer of
a. tyrosine, glutamic acid, alanine and lysine, or
b. tyrosine, phenylalanine, alanine and lysine, or
c. tryptophan; valine, alanine and lysine
wherein alanine is β-alanine (Ap) and/or β-homoalanine (Ap³); lysine is (β-lysine (Kp) and/or β-homolysine (Kp³), tyrosine is β-tyrosine (Yp) and/or β-homotyrosine (Yp³); valine is β-valine (Vp) and/or β-homovaline (Vp³); glutamic acid is β-glutamic acid (Ep) and/or β-homoglutamic acid (Ep³); phenylalanine is β-phenylalanine (Fp) and/or β-homophenylalanine (Fp³) and tryptophan is β-tryptophan (Wp) and/or β-homotryptophan (Wp).

4. The synthetic random copolymer as claimed in claim 3, wherein molecular weight of the copolymer is in the range of about 5.8 to 1 1.5 kilodaltons.

5. The synthetic random copolymer as claimed in claim 3, wherein the copolymer comprises tyrosine, glutamic acid, alanine and lysine in the molar ratio of about 1:1.5:4.3:3.3.

6. The synthetic random copolymer as claimed in claim 3, wherein the copolymer comprises tyrosine, phenylalanine, alanine and lysine in the molar ratio of about 0.5:0.5:5:3.

7. The synthetic copolymer as claimed in claim 3, wherein the copolymer comprises tryptophan, valine, alanine and lysine in the molar ratio of about 0.5:0.5:5:3.

8. The synthetic peptide or synthetic random copolymer as claimed in claim 1, exhibits increased binding affinity to multiple sclerosis associated class II MHCs (HLADR2) relative to the peptide as set forth in SEQ ID NO: 1. SEQ ID NO:2, SEQ ID NO:4 or glatiramer acetate.

9. The synthetic peptide or synthetic random copolymer as claimed in claim 1, exhibits increased binding affinity to multiple sclerosis associated class I MHCs (HLA A3) relative to the as set forth in SEQ ID NO: 1, SEQ ID NO:2, SEQ ID NO:4 or glatiramer acetate.

10. The synthetic peptide or synthetic random copolymer as claimed in claim 1, further comprises protecting groups at amino or carboxy terminus.

11. The synthetic peptide or synthetic random copolymer as claimed in claim 10, wherein protecting groups at amino terminus is selected from a group consisting of benzyloxy carbonyl, t-butyloxy carbonyl, formyl, acetyl and acyl; and protecting groups at carboxy terminus is selected from a group consisting of amides, ether and esters.

12. The synthetic peptide or synthetic random copolymer as claimed in claim 1, further comprises a label selected from the group consisting of biotin, radioisotopes, enzymes, colloidal metals or fluorescent, chemiluminescent, or phosphorescent compounds.

13. The synthetic peptide or synthetic random copolymer as claimed in claim 1, is administered subcutaneously, epicutaneously, transdermally, intramuscularly, intravenously, intraperitoneally, intrathecally, intracranially or orally in the form of a pharmaceutically acceptable salts viz. acetates, carbonates, citrate, fumerate, lactate, phosphate, glutamate, lactate, phthalate, succinate, hydrochlorides, benzathine to a subject in need thereof.

14. The synthetic peptide or synthetic random copolymer as claimed in claim 1, is administered in monomeric, oligomeric or multimeric forms to a subject in need thereof.

15. (canceled)

16. (canceled)

17. (canceled)

18. (canceled)

19. The composition as claimed in claim 18, wherein the plurality of the synthetic peptides are joined by a linker.

20. A kit comprising at least one synthetic peptide as claimed in claim 1.

21. A kit comprising at least one synthetic random copolymer as claimed in claim 4.

22. A method of ameliorating a demyelinating disorder, said method comprises administering to a subject in need thereof an effective amount of one or more peptides as claimed in claim 1.

23. (canceled)

24. A method of claim 22 wherein said subject is mammal.

25. A method of claim 22 wherein said subject is human.

26. The method as claimed in claim 22, wherein the demyelinating disorder is selected from a group consisting of multiple sclerosis (MS), optic spinal MS, Devic's disease, Acute disseminated encephalomyelitis, Balo concentric sclerosis, Schilder disease, Marburg multiple sclerosis, Guillain-Barre syndrome, chronic inflammatory, demyelinating polyneuropathy, Myalgic encephalomyelitis and Experimental autoimmune encephalomyelitis.

27. The method as claimed in claim 26, wherein the multiple sclerosis is selected from a group consisting of relapsing remitting multiple sclerosis, secondary progressive multiple sclerosis, primary progressive multiple sclerosis and chronic progressive multiple sclerosis.
